(19) United States
(12) Patent Application Publication
(10) Pub. No.: US 2025/0218588 A1
(43) Pub. Date: Jul. 3, 2025

(54) METHODS FOR DETERMINING THE PRESENCE, TYPE, OR GRADE OF A TUMOR, CYST, OR MASS, OR SUBTYPING A CANCER

(71) Applicant: The Regents of the University of California, Oakland, CA (US)

(72) Inventors: Stephen S. Francis, SAN FRANCISCO, CA (US); George Wendt, SAN FRANCISCO, CA (US); Geno Guerra, SAN FRANCISCO, CA (US)

(21) Appl. No.: 18/848,210

(22) PCT Filed: Mar. 28, 2023
(86) PCT No.: PCT/US2023/016497

§ 371 Date: Sep. 18, 2024

Related U.S. Application Data
(60) Provisional application No. 63/335,886, filed on Apr. 28, 2022.
(60) Provisional application No. 63/324,831, filed on Mar. 29, 2022.

(51) Int. Cl. G16H 50/20; C12N 15/10; G06N 20/00; G16B 30/10; G16B 35/00; G16H 50/30; G16H 50/70

(52) U.S. Cl. CPC G16H 50/20; C12N 15/1065; G06N 20/00; G16B 30/10; G16B 35/00; G16H 50/30; G16H 50/70

(57) ABSTRACT

The present disclosure is directed to methods for determining the presence, type, or grade of a tumor, cyst, lesion, mass and/or cancer, or classifying or subtyping a tumor, cyst, lesion, mass, and/or cancer, in a sample obtained from a subject.

### RELATED APPLICATION INFORMATION

This application claims priority to U.S. Application No. 63/324,831 filed on Mar. 29, 2023, and U.S. Application No. 63/335,885 filed on Apr. 28, 2022, the contents of each of which are herein incorporated by reference.

### TECHNICAL FIELD

The present disclosure relates to methods of (i) detecting or determining the presence, or type, grade, or classification of a tumor, cyst (e.g., such as a pancreatic cyst), mass, lesion, and/or cancer, or classifying or subtyping a tumor, cyst, mass, lesion, and/or cancer; or (ii) monitoring the progression or recurrence of a tumor, cyst, lesion, mass, lesion, and/or cancer in a sample obtained from a subject. The methods involve preparing an RNA sequence library comprising RNA sequences, such as full or partial RNA transcripts (e.g., mRNA, miRNA, ncRNA, rRNA, tRNA, snRNA), retroelements, transposable elements, non-coding RNA, or any combination thereof. The RNA sequence library is prepared using capture and amplification by tailing and switching from RNA isolated from extracellular vesicles from a sample obtained from a subject. Once obtained, the RNA sequences (e.g., full or partial RNA transcripts (e.g., mRNA, miRNA, ncRNA, rRNA, tRNA, snRNA), retroelements, transposable elements, non-coding RNA, or any combination thereof) are analyzed utilizing a k-mers based machine learning algorithm. The analysis of the full or partial RNA transcripts (e.g., mRNA, miRNA, ncRNA, rRNA, tRNA, snRNA), retroelements, transposable elements, non-coding RNA, or any combination thereof resulting from the k-mers based machine learning algorithm is used to (i) detect or determine the presence, type, grade or classification of a tumor, cyst, lesion, mass, and/or cancer, or classification or subtype a tumor, cyst, lesion, mass, and/or cancer; or (ii) monitor the progression or recurrence of a tumor, cyst, lesion, mass, and/or cancer in a subject.

### BACKGROUND

There are over 800,000 people living with a brain tumor in the United States. Although the majority of these are benign, over 138,000 are malignant. Around 80% of malignant brain tumors are gliomas and the most common form of glioma, glioblastoma (GBM), has a dismal 5-year survival of 5.1%¹, making GBM one of the most fatal cancers. Despite clinicians' best efforts, the treatment of gliomas has changed the survival trajectory very little in the last 15 years. The high mortality and the inherent difficulty in treating gliomas stem from the diffuse infiltration and highly aggressive nature of the tumors. Recent advances in the classification of gliomas, partially informed by the University of California San Francisco (UCSF) Adult Glioma Study (AGS), have highlighted subtype specific risk and survival which illustrate the importance of risk stratification by tumor type²⁻⁴.

Like many other cancers, standard diagnosis of most gliomas involves radiologic assessment followed by tissue biopsy. Neuroradiological evaluation of gliomas plays a critical role in both the primary diagnosis and post-therapeutic management of the disease. In high-grade gliomas (HGG) and low-grade gliomas (LGG) imaging is fundamental for monitoring tumor stability, recurrence, transformation and distinguishing between tumor recurrence and therapy-induced changes. In both HGG and LGG clinical management, including chemotherapy, anti-angiogenic therapy and radiation, can contribute to diverse post-treatment appearances making the delineation between pseudo-progression (or treatment-associated changes spanning a spectrum from acute inflammatory changes to delayed radiation necrosis) and true progression extremely challenging⁵.

Pseudo-progression, as defined by Response Assessment in Neuro-Oncology (RANO) criteria, presents as new or enlarging contrast enhancement occurring early after the completion of radiotherapy in the absence of other findings of true-progression^{6,7}. Differentiating pseudo-progression from true-progression represents a significant challenge in post treatment follow up, where misdiagnosis can lead to treatment delays from early recurrence⁵. Qualitative and quantitative (radiomic) MRI-based approaches have been employed to differentiate pseudo- from true-progression. However, the gold standard to confirm progression requires surgical biopsy that is both invasive and can have limited utility depending on the location of the tumor and the tissue heterogeneity⁸. In cases where serial imaging is favored over tissue sampling or resection, close watchful waiting takes several weeks, which further delays diagnosis and management decisions, and may not provide definitive differentiation between these processes. Currently, the only method for monitoring gliomas is radiologic assessment.

Diffuse IDH-mutant LGG are low-grade primary brain tumors that are typically diagnosed in young, otherwise healthy adults. Although most tumors initially follow an indolent clinical course, the natural history of these tumors is punctuated by repeated recurrences. A majority of patients will eventually develop high-grade transformation, resulting in rapid tumor growth and shortened survival. Median survival after transformation is just 2.4 years and early detection is shown to improve outcomes⁹. Following surgical resection of IDH-mutant LGGs, treatment strategies range from observation to aggressive treatment with radiation plus chemotherapy, or chemotherapy alone¹⁰. RTOG 9802 established a survival benefit for the addition of procarbazine, lomustine (CCNU), and vincristine (PCV) to radiotherapy over radiotherapy alone following maximal safe resection¹¹. In contemporary practice, temozolomide (TMZ) is frequently used in place of PCV due to a more favorable toxicity profile, extrapolating from trials in HGG that have demonstrated efficacy. TMZ is a cytotoxic DNA alkylating agent with mutagenic potential¹²⁻¹⁴. These agents induce cell death via mismatch repair (MMR)-mediated futile cycling, and loss of MMR is a well-described evolutionary escape mechanism for both LGG and HGG exposed to alkylators¹⁵. Cells lacking MMR function fail to recognize alkylated bases and can develop thousands of mutations distributed throughout the genome. Because of the base specificity of TMZ, these mutations are characterized by a specific mutational signature. Thus, following exposure to alkylating agents, neoplastic cells can develop defects in DNA repair that lead to a hypermutator phenotype^{12, 16-18}. It is now known that the development of hypermutation is associated with an aggressive clinical course and worse overall prognosis in transformed low grade glioma. Currently, only sequencing brain tissue can diagnose TMZ induced hypermutation.

Although no clinical liquid biopsy for glioma currently exists, glioma has been described as the “ideal candidate” for liquid biopsy due to the challenges of disease monitoring and diverse disease trajectories with personalized treatment potential¹⁹. Assessment of tumor progression and transformation using a sensitive and specific liquid biopsy alone or in conjunction with a tissue biopsy (such as, for location-restricted tumors), in conjunction with imaging, will provide neuro-oncologists with a quantitative measure to inform management potentially in near real time. Early detection of progression using liquid biopsy alone or in conjunction with a tissue biopsy would enable earlier, more informed, interventions, which would translate to improved overall outcomes as well as a reduction in the number of MRI and/or other imaging required while monitoring a patient once primary treatment begins.

Additionally, a clear benefit of near real-time monitoring of tumor progression is the ability to monitor effectiveness of given treatment in individual patients to both test novel therapies and tailor treatment. For example, if treatment A does not result in a decrease of tumor associated EV features, treatment B, C or D can be tried until an effective treatment reduces the load of tumor associated EVs. Furthermore, a non-invasive liquid biopsy to identify therapy induced hypermutation will reduce patent risk and personalize treatment approaches to improve patient outcomes. Finally, with appropriate positive predicted and negative predictive values this approach could be a suitable population level screening tool for early detection of cancer.

### SUMMARY

In one embodiment, the present disclosure relates to methods for (i) detecting or determining the presence, type, grade or classification of a tumor, cyst, lesion, mass, cancer, or any combination thereof; or (ii) classifying or subtyping a tumor, cyst, lesion, mass, cancer, or any combination thereof, in a sample obtained from a subject. The method comprises obtaining, generating and/or providing a RNA sequence library (e.g., a human RNA sequence library) from one or more samples obtained from a subject of interest (e.g., a subject of interest is a subject that has or is suspected of having cancer, a tumor, a cyst (e.g., a pancreatic cyst), a lesion, and/or mass) using capture and amplification by tailing and switching (CATS). Specifically, the sample obtained from the subject can be any type of sample provided that it contains one or more extracellular vesicles, such as, for example, blood, serum, plasma, cyst fluid, (e.g., pancreatic cyst fluid), urine, sputum, saliva, bone marrow, tears, or sweat. In some aspects, the sample is a serum sample. In other aspects, the sample is cyst fluid (e.g., pancreatic cyst fluid). The sample can be obtained using routine techniques known in the art.

RNA is isolated from the extracellular vesicles from the sample to create the RNA sequence library. More specifically, the RNA sequence library comprises RNA sequences, such as one or more retroelements, transposable elements, full or partial RNA transcripts (e.g., mRNA, miRNA, ncRNA, rRNA, tRNA, snRNA), non-coding RNA, or any combination thereof, obtained from the extracellular vesicles. Once the RNA sequence library is obtained, a processing system comprising a computer processor and a non-transitory computer memory comprising a database and at least one k-mers based machine learning algorithm is provided. The k-mers based machine learning algorithm is configured to: (i) apply the machine learning algorithm to the RNA sequence library generated previously to generate or produce k-mers results for the subject; and (ii) use the k-mers results obtained from the subject and a reference k-mers profile obtained from a control group to generate a set of probabilities to indicate whether the k-mers results from the subject are statistically similar to an outcome of interest, wherein the outcome of interest is to (i) identify the presence of a tumor, cyst, lesion, mass, cancer, or any combination thereof in the subject; (ii) determine the type or grade of tumor, cyst, lesion, mass, or cancer in the subject; (iii) classify the tumor, cyst, lesion, mass, cancer, or any combination thereof; (iv) determine the subtype of tumor, cyst, lesion, mass, cancer, or any combination thereof in the subject; or (v) any combination of (i)-(iv). Once the set of probabilities is generated, a determination is made (i) determining the presence of a tumor, cyst, lesion, mass, cancer, or any combination thereof in the subject; (ii) the type or grade of tumor, cyst, lesion, mass, and/or cancer, or any combination thereof present in the subject; (iii) the classification of the tumor, cyst, lesion, mass, cancer, or any combination thereof present in the subject; (iv) the subtype of tumor, cyst, lesion, mass, cancer, or any combination thereof in the subject; or (v) any combination of (i)-(iv).

In some aspects, the method relates to detecting or determining the presence of a tumor, cyst, lesion, mass, and/or cancer. In another aspect, the method relates to determining the type of tumor, cyst, lesion, mass, and/or cancer. In still other aspects, the method relates to determining the grade of a tumor, cyst, lesion, mass, and/or cancer in a subject. In still yet other aspects, the method relates to classifying a tumor, cyst, lesion, mass and/or cancer in a subject. In still further aspects, the method relates to subtyping or determining the subtype of a tumor, cyst, lesion, mass, and/or cancer in a subject. In some aspects, the subject is a human. In other aspects, the subject is suspected of having a tumor, cyst, lesion, mass, and/or cancer. In another aspects, the subject has a tumor, cyst, lesion, mass, and/or cancer and the subject is receiving treatment and/or being monitored in connection with said tumor, cyst, lesion, mass, and/or cancer. In another aspects, the subject previously had or suffered from a tumor, cyst, lesion, mass, and/or cancer and has finished or completed a treatment and optionally, is being monitored for recurrence of said tumor, cyst, lesion, mass, and/or cancer.

In one aspect of the above method, the tumor can be a brain tumor. In another aspect of the above method, the brain tumor is a glioma. In still yet another aspect, the glioma is an astrocytoma, glioblastoma, or oligodendroglioma. In still other aspects of the above method, the cancer can be, but is not limited to, other central nervous system tumors, meningioma, liver cancer, pancreatic cancer, colon cancer, breast cancer, bile duct cancer, kidney cancer, bladder cancer, head and neck cancers, ovarian cancer, prostate cancer, lung cancer, or any combination thereof.

In some aspects of the above method, the cysts include, but are not limited to, acne cysts, arachnoid cysts, Baker's cysts, Bartholin's cysts, breast cysts, chalazion cysts, colloid cysts, dentigerous cysts, dermoid cysts, epididymal cysts, ganglion cysts, hydatid cysts, kidney cysts, ovarian cysts, pancreatic cysts, periapical cysts, pilar cysts, pilonidal cysts, pineal gland cysts, sebaceous cysts, tarlov cysts, vocal fold cysts, or any combination thereof. In some aspects, the cyst is a pancreatic cyst or PCL. In some further aspects, the method comprises determining the type of pancreatic cyst. In still other embodiments, the method comprises classifying the type of pancreatic cyst (e.g., low grade versus high grade, benign pancreatic cyst from a pancreatic cyst having malignant potential).

In yet some further aspects of the above method, the method further comprises obtaining a sample from the subject (any type of sample obtained from a subject can be used provided that it contains one or more extracellular vesicles, such as, for example, blood, serum, plasma, cyst fluid, (e.g., pancreatic cyst fluid), urine, sputum, saliva, bone marrow, tears, or sweat) and isolating extracellular vesicles in the sample. The sample can be obtained from the subject using any techniques known in the art. In some aspects, the sample is a serum sample. In other aspects, the sample is a plasma sample. In still other aspects, the sample is a cyst fluid sample.

In other aspects of the above method, the capture by amplification and tail switching (CATS) library preparation is modified utilizing polyethylene glycol molecular crowding to increase the efficiency of RNA sequencing. In further aspects of the method, the modified CATS library preparation utilizes unique molecular identifiers (UMI), where random base pairs are synthesized on sequence adapters to aid in direct quantification of the RNA template.

In other aspects of the above method, the CATS method is modified to function with extremely low RNA input by utilizing polyethylene glycol crowding, custom oligo alterations to increase template switching efficiency, unique molecular identifiers (UMI), and combination thereof to allow for direct quantification of each RNA molecule. In some aspects of the above method, the RNA sequences are one or more retroelements, transposable elements, full or partial RNA transcripts (e.g., mRNA, miRNA, ncRNA, rRNA, tRNA, snRNA), non-coding RNA, or any combination thereof. In some aspects, the retroelements and/or transposable elements include long interspersed nuclear elements (LINE), short interspersed nuclear elements (SINE), SINE-VNTR-Alu (SVA), long terminal repeat (LTR) retroelements, non-LTR elements, Tyrosine recombinase (YR) retroelements, Penelope like elements (PLEs), pericentromeric satellites, alpha satellites, or any combination thereof.

In another embodiment, the present disclosure relates to monitoring progression or recurrence of a tumor, cyst, lesion, mass, cancer, or any combination thereof in a subject. The method comprises obtaining, generating and/or providing a RNA sequence library (e.g., a human RNA sequence library) from one or more samples obtained from a subject of interest (e.g., a subject of interest is a subject that has or has previously had cancer, a tumor, a cyst (e.g., a pancreatic cyst), a lesion and/or mass) using capture and amplification by tailing and switching (CATS). In some aspects, the sample is any type of sample that is obtained from a subject provided that it contains one or more extracellular vesicles, such as, for example, blood, serum, plasma, cyst fluid, (e.g., pancreatic cyst fluid), urine, sputum, saliva, bone marrow, tears, or sweat. RNA is isolated from the extracellular vesicles from the sample to create the RNA sequence library, using routine techniques known in the art. More specifically, the RNA sequence library comprises RNA sequences, such as one or more retroelements and/or transposable elements obtained from the extracellular vesicles. Once the RNA sequence library is obtained, a processing system comprising a computer processor and a non-transitory computer memory comprising a database and at least one k-mers based machine learning algorithm is provided. The k-mers based machine learning algorithm is configured to: (i) apply the machine learning algorithm to the RNA sequence library generated previously to generate or produce k-mers results for the subject; and (ii) use the k-mers results obtained from the subject and a reference k-mers profile obtained from a control group, to generate a set of probabilities to indicate whether the k-mers results from the subject are statistically similar to an outcome of interest, wherein the outcome of interest is to identify whether (i) the tumor, cyst, lesion, mass, cancer, or any combination thereof in the subject has increased in size and progressed or decreased in size (e.g., which may indicate the efficacy of the treatment); or (ii) the tumor, cyst, lesion, mass, cancer, or any combination thereof has reoccurred or re-appeared in the subject.

Once the set of probabilities is generated, a determination is made whether (i) the tumor, cyst, lesion, mass, cancer, or any combination thereof in the subject has increased in size and progressed or decreased in size; or (ii) the tumor, cyst, lesion, mass, cancer, or any combination thereof has reoccurred or re-appeared in the subject.

In some aspects of the above method, the method further comprises predicting the survival of the subject based on the determination of whether the tumor, cyst, lesion, mass, cancer, or any combination thereof has or has not progressed in the subject of interest. In another aspect, the subject of interest has a tumor, cyst, lesion, mass, cancer, or any combination thereof and the subject is receiving treatment and/or being monitored for said tumor, cyst, lesion, mass, cancer, or any combination thereof. In another aspects, the subject of interest previously had or suffered from a tumor, cyst, lesion, mass, cancer, or any combination thereof and has finished or completed a treatment and optionally, is being monitored for recurrence of said tumor, cyst, lesion, mass, cancer, or any combination thereof.

In yet other aspects of the above method, the tumor can be a brain tumor. In another aspect of the above method, the brain tumor is a glioma. In still yet another aspect, the glioma is an astrocytoma, glioblastoma, or oligodendroglioma. In still other aspects, the cancer can be, but is not limited to, other central nervous system tumors, meningioma, liver cancer, pancreatic cancer, colon cancer, breast cancer, bile duct cancer, kidney cancer, bladder cancer, head and neck cancers, ovarian cancer, prostate cancer, lung cancer, or any combination thereof.

In some aspects of the above method, the cysts can be, but not are not limited to, acne cysts, arachnoid cysts, Baker's cysts, Bartholin's cysts, breast cysts, chalazion cysts, colloid cysts, dentigerous cysts, dermoid cysts, epididymal cysts, ganglion cysts, hydatid cysts, kidney cysts, ovarian cysts, pancreatic cysts, periapical cysts, pilar cysts, pilonidal cysts, pineal gland cysts, sebaceous cysts, tarlov cysts, vocal fold cysts, or any combination thereof. In yet other aspects of the above method, the cyst is a pancreatic cyst or PCL.

In yet some further aspects of the above method, the method further comprises obtaining sample from the subject and isolating extracellular vesicles in the sample. In some aspects, any type of sample obtained from a subject can be used provided that it contains one or more extracellular vesicles, such as, for example, blood, serum, plasma, cyst fluid, (e.g., pancreatic cyst fluid), urine, sputum, saliva, bone marrow, tears, or sweat. In some aspects, the sample is a serum sample. In other aspects, the sample is cyst fluid (e.g., pancreatic cyst fluid). The sample can be obtained using routine techniques known in the art.

In other aspects of the above method, the capture by amplification and tail switching (CATS) library preparation is modified utilizing polyethylene glycol molecular crowding to increase the efficiency of RNA sequencing. In further aspects of the method, the modified CATS library preparation utilizes unique molecular identifiers (UMI), where random base pairs are synthesized on sequence adapters to aid in direct quantification of the RNA template.

In other aspects of the above method, the CATS method is modified to function with extremely low RNA input by utilizing polyethylene glycol crowding, custom oligo alterations to increase template switching efficiency, unique molecular identifiers (UMI), and combination thereof to allow for direct quantification of each RNA molecule.

In some aspects of the above method, the RNA sequences are one or more retroelements, transposable elements, full or partial RNA transcripts (e.g., mRNA, miRNA, ncRNA, rRNA, tRNA, snRNA), non-coding RNA, or any combination thereof. In some aspects, the retroelements and/or transposable elements include long interspersed nuclear elements (LINE), short interspersed nuclear elements (SINE), SINE-VNTR-Alu (SVA), long terminal repeat (LTR) retroelements, non-LTR elements, Tyrosine recombinase (YR) retroelements, Penelope like elements (PLEs), pericentromeric satellites, alpha satellites, or any combination thereof.

In yet a further embodiment, the present disclosure relates to methods for diagnosing a glioma in a subject. The method comprises generating and/or providing a RNA sequence library (e.g., a human RNA sequence library) from one or more samples obtained from a subject of interest (e.g., a subject of interest is a subject that has or is suspected of having a cancer and/or a glioma) using capture and amplification by tailing and switching (CATS). Specifically, any type of sample obtained from a subject can be used provided that it contains one or more extracellular vesicles, such as, for example, blood, serum, plasma, cyst fluid, (e.g., pancreatic cyst fluid), urine, sputum, saliva, bone marrow, tears, or sweat. In some aspects, the sample is a serum sample. In other aspects, the sample is cyst fluid (e.g., pancreatic cyst fluid). The sample can be obtained using routine techniques known in the art.

RNA is isolated from the extracellular vesicles from the sample to create the RNA sequence library. The sample can be obtained using routine techniques known in the art. More specifically, the RNA sequence library comprises RNA sequences, such as one or more retroelements and/or transposable elements obtained from the extracellular vesicles. Once the RNA sequence library is obtained, a processing system comprising a computer processor and a non-transitory computer memory comprising a database and at least one k-mers based machine learning algorithm is provided. The k-mers based machine learning algorithm is configured to: (i) apply the machine learning algorithm to the RNA sequence library generated previously to generate or produce k-mers results for the subject; and (ii) use the k-mers results obtained from the subject and a reference k-mers profile obtained from a control group, to generate a set of probabilities to indicate whether the k-mers results from the subject are statistically similar to an outcome of interest, wherein the outcome of interest is to identify the presence or absence of a glioma in the subject. Once the set of probabilities is generated, a determination is made whether or not the subject has a glioma.

In the above method, the glioma is an astrocytoma, glioblastoma, or oligodendroglioma.

In other aspects of the above method, the capture by amplification and tail switching (CATS) library preparation is modified utilizing polyethylene glycol molecular crowding to increase the efficiency of RNA sequencing. In further aspects of the method, the modified CATS library preparation utilizes unique molecular identifiers (UMI), where random base pairs are synthesized on sequence adapters to aid in direct quantification of the RNA template.

In other aspects of the above method, the CATS is modified to function with extremely low RNA input by utilizing polyethylene glycol crowding, custom oligo alterations to increase template switching efficiency, unique molecular identifiers (UMI), and combination thereof to allow for direct quantification of each RNA molecule.

In some aspects of the above method, the RNA sequences are one or more retroelements and/or transposable elements. In some aspects, the retroelements, transposable elements, full or partial RNA transcripts (e.g., mRNA, miRNA, ncRNA, rRNA, tRNA, snRNA) non-coding RNA, or any combination thereof include long interspersed nuclear elements (LINE), short interspersed nuclear elements (SINE), SINE-VNTR-Alu (SVA), long terminal repeat (LTR) retroelements, non-LTR elements, Tyrosine recombinase (YR) retroelements, Penelope like elements (PLEs), pericentromeric satellites, alpha satellites, or any combination thereof.

In another embodiment, the present disclosure relates to a system for (i) detecting or determining the presence, type, or grade of a tumor, cyst, lesion, mass, cancer, or any combination thereof; or (ii) classifying or subtyping a tumor, cyst, lesion, mass, cancer, or any combination thereof. The system comprises: (a) a RNA sequence library using capture and amplification by tailing and switching (CATS) from RNA isolated from extracellular vesicles from a sample obtained from a subject, wherein the RNA sequence library comprises RNA sequences, such as one or more retroelements, transposable elements or combination thereof, from the RNA isolated from the extracellular vesicles; (b) a k-mers based machine learning algorithm for analyzing the RNA sequences (e.g., on one or more retroelements, transposable elements, full or partial RNA transcripts (e.g., mRNA, miRNA, ncRNA, rRNA, tRNA, snRNA), non-coding RNA, or any combination thereof) from the RNA sequence library obtained from a subject; and (c) a reference database from control subjects for detecting or determining the presence, type, or grade of the tumor, cyst, lesion, mass, and/or cancer, or classifying or subtyping the tumor, cyst, lesion, mass and/or cancer in the sample based on the analysis in (b).

In one aspect of the above system, the sample obtained from a subject is any type of sample provided that it contains one or more extracellular vesicles, such as, for example, blood, serum, plasma, cyst fluid, (e.g., pancreatic cyst fluid), urine, sputum, saliva, bone marrow, tears, or sweat. In some aspects, the sample is a serum sample. In other aspects, the sample is cyst fluid (e.g., pancreatic cyst fluid). The sample can be obtained using routine techniques known in the art.

In another aspect of the above system, the tumor can be a brain tumor. In another aspect of the above system, the brain tumor is a glioma. In still yet another aspect, the glioma is an astrocytoma, glioblastoma, or oligodendroglioma. In still other aspects, the cancer can be, but not limited to, other central nervous system tumors, meningioma, liver cancer, pancreatic cancer, colon cancer, breast cancer, bile duct cancer, kidney cancer, bladder cancer, head and neck cancers, ovarian cancer, prostate cancer, lung cancer, or any combination thereof.

In some aspects of the above system, the cysts can be, but are not limited to, acne cysts, arachnoid cysts, Baker's cysts, Bartholin's cysts, breast cysts, chalazion cysts, colloid cysts, dentigerous cysts, dermoid cysts, epididymal cysts, ganglion cysts, hydatid cysts, kidney cysts, ovarian cysts, pancreatic cysts, periapical cysts, pilar cysts, pilonidal cysts, pineal gland cysts, sebaceous cysts, tarlov cysts, vocal fold cysts, or any combination thereof. In yet other aspects of the above system, the cyst is a pancreatic cyst.

In the above system, the serum can be a liquid biopsy collected from a glioma resection.

In other aspects of the above system, the capture by amplification and tail switching (CATS) library preparation is modified by utilizing polyethylene glycol molecular crowding to increase the efficiency of RNA sequencing. In further aspects of the system, the modified CATS library preparation utilizes unique molecular identifiers (UMI), where random base pairs are synthesized on sequence adapters to aid in direct quantification of the RNA template.

In some aspects of the above system, the RNA sequences are one or more retroelements, transposable elements, full or partial RNA transcripts (e.g., mRNA, miRNA, ncRNA, rRNA, tRNA, snRNA), non-coding RNA, or any combination thereof. In some aspects, the retroelements and/or transposable elements include long interspersed nuclear elements (LINE), short interspersed nuclear elements (SINE), SINE-VNTR-Alu (SVA), long terminal repeat (LTR) retroelements, non-LTR elements, Tyrosine recombinase (YR) retroelements, Penelope like elements (PLEs), pericentromeric satellites, alpha satellites, or any combination thereof.

In yet a further embodiment, the present disclosure relates to methods of improving the accuracy of determining whether a subject is at risk of developing a glioma or a recurrence of a glioma. The method comprises generating and/or providing a RNA sequence library (e.g., a human RNA sequence library) from one or more samples obtained from a subject of interest (e.g., a subject of interest is a subject that has or is suspected of having a cancer and/or a glioma or previously had cancer or a glioma and is suspected of re-occurrence or reappearance of the cancer or glioma) using capture and amplification by tailing and switching (CATS).

Specifically, the method further comprises obtaining a sample from the subject (any type of sample obtained from a subject can be used provided that it contains one or more extracellular vesicles, such as, for example, blood, serum, plasma, cyst fluid, (e.g., pancreatic cyst fluid), urine, sputum, saliva, bone marrow, tears, or sweat) and isolating extracellular vesicles in the sample. In some aspects, the sample is a serum sample. In other aspects, the sample is a plasma sample. The sample can be obtained from the subject using any techniques known in the art. In some aspects, the sample is a serum sample. In other aspects, the sample is a plasma sample.

RNA is isolated from the extracellular vesicles from the sample to create the RNA sequence library. More specifically, the RNA sequence library comprises RNA sequences, such as one or more retroelements, transposable elements, full or partial RNA transcripts (e.g., mRNA, miRNA, ncRNA, rRNA, tRNA, snRNA), non-coding RNA, or any combination thereof, obtained from the extracellular vesicles. Once the RNA sequence library is obtained, the sequences in the sequence library are aligned with a reference genome sequence (e.g., such as obtained from a control group). After the sequences from the RNA sequence library (e.g., full or partial RNA transcripts (e.g., mRNA, miRNA, ncRNA, rRNA, tRNA, snRNA), retroelements, transposable elements, non-coding RNA, or any combination thereof) from the subject are aligned with the reference genome sequence, a processing system comprising a computer processor and a non-transitory computer memory comprising a database and at least one k-mers based machine learning algorithm is provided. The k-mers based machine learning algorithm is configured to: (i) apply the machine learning algorithm to the sequences from the RNA library (e.g., retroelements, transposable elements, full or partial RNA transcripts (e.g., mRNA, miRNA, ncRNA, rRNA, tRNA, snRNA), non-coding RNA, or combination thereof) aligned previously to generate or produce k-mers results for the subject; and (ii) use the k-mers results obtained from the subject and a reference k-mers profile obtained from a reference or control group, to generate a set of probabilities to indicate whether the k-mers results from the subject are statistically similar to an outcome of interest, wherein the outcome of interest is to determine whether or not the subject is at risk of developing a glioma or a re-occurrence or reappearance of a glioma. Once the set of probabilities is generated, a determination is made whether (or not) the subject is at risk of developing a glioma or that a glioma has reoccurred or re-appeared in the subject.

In one aspect of the above method the reference genome sequence is hg38 or hg19.

In another aspect of the above method, the glioma is an astrocytoma, glioblastoma, or oligodendroglioma.

In other aspects of the above method, the capture by amplification and tail switching (CATS) library preparation is modified utilizing polyethylene glycol molecular crowding to increase the efficiency of RNA sequencing. In further aspects of the method, the modified CATS library preparation utilizes unique molecular identifiers (UMI), where random base pairs are synthesized on sequence adapters to aid in direct quantification of the RNA template.

In some aspects of the above method, the RNA sequences are one or more retroelements, transposable elements, full or partial RNA transcripts (e.g., mRNA, miRNA, ncRNA, rRNA, tRNA, snRNA), non-coding RNA, or any combination thereof. In some aspects, the retroelements and/or transposable elements include long interspersed nuclear elements (LINE), short interspersed nuclear elements (SINE), SINE-VNTR-Alu (SVA), long terminal repeat (LTR) retroelements, non-LTR elements, Tyrosine recombinase (YR) retroelements, Penelope like elements (PLEs), pericentromeric satellites, alpha satellites, or any combination thereof.

In yet another embodiment, the present disclosure relates to method of improving the accuracy of determining whether a subject is at risk of developing a glioma or re-occurrence or reappearance of a glioma. The method comprises: (a) generating a sequence library from RNA isolated from extracellular vesicles obtained from a sample of a subject, wherein the sequence library comprises RNA of one or more full or partial RNA transcripts (e.g., mRNA, miRNA, ncRNA, rRNA, tRNA, snRNA), retroelements, transposable elements, non-coding RNA, or any combination thereof, obtained from the extracellular vesicles using capture and amplification by tailing and switching (CATS) and one or more unique molecular identifiers; (b) aligning the sequences of the RNA sequence library (containing one or more retroelements, one or more transposable elements, or combination thereof) generated in step a) with a reference genome sequence; (c) providing a processing system comprising a computer processor and a non-transitory computer memory comprising a database and at least one k-mers based machine learning algorithm, wherein the k-mers based machine learning algorithm is configured to: (i) apply the machine learning algorithm to the RNA sequences aligned in step b) to generate k-mers results for the subject; and (ii) use the k-mers results from the subject and a reference k-mers profile obtained from a control group to generate a set of probabilities to indicate whether the k-mers results from the subject are statistically similar to an outcome of interest, wherein the outcome of interest is to identify (i) whether the subject is at risk of developing a glioma; or (ii) re-occurrence or re-appearance of a glioma; and (d) determining whether (i) the subject is at risk of a glioma; or (ii) whether or not a glioma has reoccurred or re-appeared in the subject based on the probabilities generated in step c).

In the above method, the sample obtained from the subject can be any type of sample provided that it contains one or more extracellular vesicles, such as, for example, blood, serum, plasma, cyst fluid, (e.g., pancreatic cyst fluid), urine, sputum, saliva, bone marrow, tears, or sweat). The sample can be obtained from the subject using any techniques known in the art. In some aspects, the sample is a serum sample. In other aspects, the sample is a plasma sample. The sample can be obtained using routine techniques known in the art.

In one aspect of the above method, the reference genome sequence is hg38 or hg19. In another aspect of the above method, the glioma is an astrocytoma, glioblastoma, or oligodendroglioma. In other aspects of the above method, the capture by amplification and tail switching (CATS) library preparation is modified utilizing polyethylene glycol molecular crowding to increase the efficiency of RNA sequencing. In further aspects of the method, the modified CATS library preparation utilizes unique molecular identifiers (UMI), where random base pairs are synthesized on sequence adapters to aid in direct quantification of the RNA template.

### BRIEF DESCRIPTION OF THE FIGURES

FIG. 1 shows experimental design for selecting EV RNA library preparation for glioma prediction.

FIG. 2 shows GlioEV results of subtype prediction. Further, it shows PC's of machine learning prediction model and 10-fold cross validation accuracy.

FIG. 3 shows that retroelement ALR/Alpha is predictive of IDH status levels in serum EV's and exhibits similar differential expression in TCGA tumor.

FIG. 4 shows differential expression of EV RNA from cyst fluid in LGD (pink) and HGD/AN (turquoise), identical isolation and sequencing protocol as described in Aim1. All RNA features are significant at an FDR of 0.05 between LGD vs HGD/AN. LINE-1 elements dominate upregulation in AN samples. Perfect hierarchical clustering from retroelements and near prefect hierarchical clustering from mRNA (genes).

FIG. 5 shows the results of k-mer machine learning trained on cyst EV RNA. Prediction accuracy assessed by 10-fold leave one out cross validation. FIG. 5A shows PCA of RNA features used in prediction model. FIG. 5B shows the prediction of HGD/AN subjects. FIG. 5C shows the prediction of LGD subjects.

### III. DETAILED DESCRIPTION

### Definitions

Unless otherwise defined, all technical and scientific terms used herein have the same meaning as commonly understood by one of ordinary skill in the art to which this disclosure belongs. Methods and materials are described herein for use in the present disclosure; other, suitable methods and materials known in the art can also be used. The materials, methods, and examples are illustrative only and not intended to be limiting.

As used herein, the term “about” or “approximately” means within an acceptable error range for the particular value as determined by one of ordinary skill in the art, which will depend in part on how the value is measured or determined, (i.e., the limitations of the measurement system). For example, “about” can mean within 1 or more than 1 standard deviations, per practice in the art. Where particular values are described in the application and claims, unless otherwise stated, the term “about” means within an acceptable error range for the particular value.

As used herein, the terms “comprise(s),” “include(s),” “having,” “has,” “can,” “contain(s),” and variants thereof, are intended to be open-ended transitional phrases, terms, or words that do not preclude the possibility of additional acts or structures. The singular forms “a,” “and,” and “the” include plural references unless the context clearly dictates otherwise. The present disclosure also contemplates other embodiments “comprising,” “consisting of” and “consisting essentially of,” the embodiments or elements presented herein, whether explicitly set forth or not.

As used herein, the phrase “capture and amplification by tailing and switching (CATS)” refers to a ligation-independent method for generating ready-to-sequence DNA libraries from low amounts (e.g., picogram amounts) of either DNA or RNA molecules for next generation sequencing. An example of a CATS method that can be used in the present disclosure is the method described in Turchinovich A, Surowy H, Serva A, Zapatka M, Lichter P, Burwinkel B., “Capture and Amplification by Tailing and Switching (CATS). An ultrasensitive ligation-independent method for generation of DNA libraries for deep sequencing from picogram amounts of DNA and RNA”, RNA Biol., 2014; 11 (7): 817-28, the contents of which are herein incorporated by reference.

As used herein the term “cancer” refers to a disease or condition in which some of the body's cells grow uncontrollably and spread to other parts of the body. Many cancers form solid tumors, but cancers of the blood, such as leukemias, generally do not. There are more than 100 types of cancer. Types of cancer are usually named for the organs or tissues where the cancers form. For example, lung cancer starts in the lung, and brain cancer starts in the brain. Cancers also may be described by the type of cell that formed them, such as an epithelial cell or a squamous cell. Categories of cancers that begin in specific types of cells include: (a) carcinomas; (b) sarcomas; (c) leukemias; (d) lymphoma (e) multiple myeloma; (f) melanoma; and/or (g) brain and spinal cord tumors. Examples of carcinomas include breast cancer, colon cancer, prostate cancer, bladder cancer, lung cancer, stomach cancer, kidney cancer, and intestines cancer. Sarcomas are cancers that form in bone and soft tissues, including muscle, fat, blood vessels, lymph vessels, and fibrous tissue (such as tendons and ligaments), and include osteosarcoma, leiomyosarcoma, Kaposi sarcoma, malignant fibrous histiocytoma, liposarcoma, and dermatofibrosarcoma protuberans. Leukemias are cancers that begin in the blood-forming tissues of the bone marrow. Lymphoma includes Hodgkin lymphoma and non-Hodgkin lymphoma. Multiple myeloma is a cancer that begins in plasma cells. Melanoma is cancer that begins in cells that become melanocytes, which are specialized cells that make melanin (such as the pigment that gives skin its color).

As used herein, the term “cyst” refers to a sac-like pocket of membranous tissue that contains fluid, air, or other substances. Cysts can grow almost anywhere in a subject's body or under the skin. Most cysts are benign, or noncancerous and develop due to blockages in the body's natural drainage systems. However, some cysts are tumors that form inside tumors. Cysts can be malignant, or cancerous. Examples of cysts include, but are not limited to, cystic acne, or nodulocystic acne; arachnoid cysts; Baker's cysts, which are also called popliteal cysts; Bartholin's cysts; breast cysts; chalazion cysts; dentigerous cysts; epididymal cysts or spermatoceles; ganglion cysts; hydatid cysts; kidney cyst or renal cyst; ovarian cysts; pancreatic cysts; periapical cysts, which are also known as radicular cysts; pilar cysts, which are also known as trichilemmal cysts; pilonidal cysts; pineal gland cysts; sebaceous cysts; tarlov cysts, which are also known as perineural, perineurial, or sacral nerve root cysts; and vocal fold cysts, such as mucus retention cysts and epidermoid cysts. In some aspects, the cyst is a pancreatic cyst. In some aspects, the cyst is a pancreatic cyst or pancreatic cyst lesion (PCL).

As used herein the term “extracellular vesicles (EVs)” refers to membrane bound vesicles secreted from almost all types of cells into the extracellular space. Unlike most types of cells, EVs cannot replicate. The three main subtypes of EVs are microvesicles (MVs), exosomes, and apoptotic bodies, which are differentiated based upon their biogenesis, release pathways, size, content, and function. Extracellular vesicles come in a variety of sizes and range in diameter from about 20 nanometers to about 10 microns or more, although, the vast majority of EVs are smaller than about 200 nm.

As used herein the term “glioma” refers to a type of tumor that occurs in the brain and spinal cord. Examples of gliomas include: astrocytomas, including astrocytoma, anaplastic astrocytoma and glioblastoma; ependymomas, including anaplastic ependymoma, myxopapillary ependymoma and subependymoma; and oligodendrogliomas, including oligodendroglioma, anaplastic oligodendroglioma and anaplastic oligoastrocytoma. Gliomas are one of the most common types of primary brain tumors.

As used herein, the phrase “interactive multi-objective k-mer analysis (iMOKA)” refers to a software that enables comprehensive analysis of sequencing data from large cohorts to generate robust classification models or explore specific genetic elements associated with disease etiology. iMOKA uses a fast and accurate feature reduction step that combines a Naïve Bayes classifier augmented by an adaptive entropy filter and a graph-based filter to rapidly reduce the search space. By using a flexible file format and distributed indexing, iMOKA can easily integrate data from multiple experiments and also reduces disk space requirements and identifies changes in transcript levels and single nucleotide variants. An example of iMOKA that can be used in the present disclosure is that described in Lorenzi C, Barriere S, Villemin J P, Dejardin Bretones L, Mancheron A, Ritchie W., “iMOKA: k-mer based software to analyze large collections of sequencing data”, Genome Biol., 2020; 21 (1): 261, the contents of which are herein incorporated by reference.

As used herein, the term “k-mers” refers to substrings of a length k contained within a biological sequence. K-mers are primarily used within the context of computational genomics and sequence analysis, in which k-mers are composed of nucleotides (i.e., A, T/U, G, and C). In some aspects, the term k-mer refers to all of a se'uence's subsequences of a length k, such that the sequence AGAT would have four monomers (A, G, A, and T/U), three 2-mers (AG, GA, AT/U), two 3-mers (AGA and GAT/U) and one 4-mer (AGAT/U). More generally, a sequence of length L will have L-k+1 k-mers and nk total possible k-mers, where n is number of possible monomers (e.g., four in the case of DNA).

As used herein the term “mass” refers to a lump in the body of a subject. A mass may be caused by the abnormal growth of cells, a cyst, hormonal changes, or an immune reaction. A mass may be benign (not cancerous) or malignant (cancerous).

As used herein, the term “retroelement” refers to mobile genetic elements (MGEs) that in some cases retrotranspose via an RNA intermediate that is reverse-transcribed to DNA and integrated into a new location within the host or subject genome. Retroelements have been found among different organisms from bacteria to humans and often constitute a significant part of genomes, particularly in higher plants and fungi. Examples of retroelements include LINE (Long Interspersed Element), SINE (Short Interspersed Elements, such as Alu elements), ALR/Alpha, long terminal repeats (LTRs) containing elements, non-LTR elements, Tyrosine recombinase (YR) elements, Penelope retrotransposons (PLEs) or any combination thereof.

“Subject” and “patient” as used herein interchangeably refers to any vertebrate, including, but not limited to, a mammal (e.g., cow, pig, camel, llama, horse, goat, rabbit, sheep, hamsters, guinea pig, cat, dog, rat, and mouse, a non-human primate (for example, a monkey, such as a cynomolgous or rhesus monkey, chimpanzee, etc.) and a human). In some aspects, the subject may be a human or a non-human. In some aspects, the subject is a human. The subject or patient may be undergoing other forms of treatment. In some aspects, the subject is a human that may be undergoing other forms of treatment.

As used herein, the phrase “subtyping a cancer” refers to the smaller groups that a type of cancer can be divided into, based on certain characteristics of the cancer cells. These characteristics include how the cancer cells look under a microscope and whether there are certain substances in or on the cells or certain changes to the DNA of the cells. Subtyping of a cancer is important in order to plan treatment and determine prognosis.

As used herein, the term “tumor” refers to any abnormal mass of tissue that forms when cells grow and divide more than they should or do not die when they should. Tumors may be benign (not cancer) or malignant (cancer). Noncancerous tumors can become cancerous if not treated. Examples of malignant (cancerous) tumors include: (i) bone tumors, such as osteosarcoma and chordomas; (ii) brain tumors such as glioblastoma and astrocytoma; (iii) malignant soft tissue tumors and sarcomas; (iv) organ tumors such as lung cancer and pancreatic cancer; (v) ovarian germ cell tumors; and/or (vi) skin tumors, such as squamous cell carcinoma. Examples of benign (noncancerous) tumors include: (i) benign bone tumors such as osteomas; (ii) brain tumors such as meningiomas and schwannomas; (iii) gland tumors such as pituitary adenomas; (iv) lymphatic tumors such as angiomas; (v) benign soft tissue tumors such as lipomas; and/or (vi) uterine fibroids. Type of precancerous tumors include: (i) actinic keratosis, a type of skin condition; (ii) cervical dysplasia; (iii) colon polyps; and/or (iv) ductal carcinoma in situ, a type of breast tumor.

As used herein, the phrase “tumor grade” refers to the description of a tumor based on appearance cancer cells and tissue, namely, how abnormal the tumor cells and the tumor tissue look under a microscope. It is an indicator of how quickly a tumor is likely to grow and spread.

As used herein, the phrase “unique molecular identifiers (UMIs)” refers to a type of molecular barcoding that provides error correction and increased accuracy during sequencing. Molecular barcodes can comprise short sequences that are used to uniquely tag each molecule in a sample library. UMIs are used for a wide range of sequencing applications, such as identifying PCR errors (e.g., Because the nucleic acid in the starting material is tagged with a unique molecular barcode, bioinformatics software can filter out duplicate reads and PCR errors with a high level of accuracy and report unique reads, removing the identified errors before final data analysis). UMI deduplication is also useful for RNA-sequence gene expression analysis and other quantitative sequencing methods.

II. Methods for (i) Detecting or Determining the Presence, Type, or Grade of a Tumor, Cyst, Lesion, Mass, Cancer, or any Combination Thereof; or (ii) Classifying or Subtyping a Tumor, Cyst, Lesion, Mass, Cancer, or any Combination Thereof in a Sample from a Subject

In one embodiment, the present disclosure relates to methods for (i) detecting or determining the presence, type, or grade of a tumor, cyst, lesion, mass, cancer, or any combination thereof; or (ii) classifying or subtyping a tumor, cyst, lesion, mass, cancer, or any combination thereof in a sample obtained from a subject. Specifically, the methods of the present disclosure comprise preparing, generating, obtaining, and/or providing a RNA sequence library using capture and amplification by tailing and switching (CATS) from RNA isolated from extracellular vesicles obtained from a sample of a subject of interest using routine techniques known in the art. In some aspects, a “subject of interest” refers to a subject that has or is suspected of having a tumor, cyst, lesion, mass, cancer, or any combination thereof. In some aspects, the RNA sequence library comprises RNA sequences, such as, at least one or more retroelements, transposable elements, full or partial RNA transcripts (e.g., mRNA, miRNA, ncRNA, rRNA, tRNA, snRNA), non-coding RNA, or any combination thereof that are obtained from the RNA isolated from the extracellular vesicles.

Once a RNA sequence library is generated, the RNA sequences (e.g., retroelements, transposable elements, full or partial RNA transcripts (e.g., mRNA, miRNA, ncRNA, rRNA, rRNA, snRNA), non-coding RNA, or combination thereof) are analyzed utilizing a k-mers based machine learning algorithm. Specifically, the k-mers based machine learning algorithm is configured to first apply the machine learning algorithm to the RNA sequence library generated previously to generate or produce k-mers results for the subject of interest (“subject k-mers results”).

In some aspects of the above method, the accuracy of method can be improved by prior to performing or utilizing the k-mers based machine learning algorithm, aligning the RNA sequences in the RNA sequence library with a reference genome sequence using routine techniques known in the art (such as by using a short read aligner such as BowTie, BWA or STAR). These alignments are then collapsed by UMI to accurately quantify the number of unique RNA molecules sequenced. In some aspects, when determining whether a subject has a glioma, reference genomes such as hg38 or hg19 can be used.

In some aspects, the RNA sequences (e.g., retroelement, transposable elements, full or partial RNA transcripts (e.g., mRNA, miRNA, ncRNA, rRNA, tRNA, snRNA), non-coding RNA, or any combination thereof), align with the reference genome with at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, or at least 100% sequence identity.

In some aspects, a consensus sequence is generated from the alignment of the RNA sequences with the reference genome, and unique molecular indicators (UMIs). Without wishing to be bound by any theory, the inclusion of this alignment (of the RNA sequences with the reference genome) increases the accuracy of the method by reducing bias from sequencing and/or random error. More specifically, it was found that the use of UMIs in the CATS library preparation could introduce error into the analysis by miscalling polymorphisms in the sequencing due to error. As a result, this created errors in the k-mer analysis that were incorrect due to a single base error. By aligning the RNA sequences from the RNA sequence library (such as one or more full or partial RNA transcripts (e.g., mRNA, miRNA, ncRNA, rRNA, tRNA, snRNA), retroelements, transposable elements, non-coding RNA, or any combination thereof), with a reference genome sequence and utilizing a consensus sequence, the comparability of the k-mers being compared is ensured and the accuracy of the method is increased. Once this alignment is completed, the RNA sequences (e.g., full or partial RNA transcripts (e.g., mRNA, miRNA, ncRNA, rRNA, tRNA, snRNA), retroelements, transposable elements, non-coding RNA, or any combination thereof) can be used in the k-mers based machine learning algorithm to generate the subject's k-mers results.

Once the subject k-mers results are generated, these results are obtained and reported by the algorithm. After reporting, the subject k-mers results are analyzed against a reference k-mers profile. Specifically, the reference k-mers profile is a set of results obtained from a suitable control group. A suitable control group for use in the methods described herein can be determined and obtained using routine techniques known in the art.

Once the reference k-mers profile is provided, the k-mers based machine learning algorithm compares the subject k-mers results with those of the reference k-mers profile to generate a set of probabilities to indicate whether the subject k-mers results are statistically similar to an outcome of interest. This set of probabilities can be communicated (e.g., reported) for further analysis, interpretation, processing and/or display. The result can be communicated (e.g., reported) by the system, such as by a computer, in a document and/or spreadsheet, on a mobile device (e.g., a smart phone), on a website, in an e-mail, or any combination thereof. In some aspects, the set of probabilities are used by a clinician to determine an outcome of interest.

In some aspects, the outcome of interest is to (i) detect and/or identify the presence of a tumor, cyst, lesion, mass, or cancer in the subject; (ii) determine the type or grade of tumor, cyst, lesion, mass, or cancer in the subject; (iii) classify the tumor, cyst, lesion, mass, cancer, or any combination thereof in the subject; (iv) determine the subtype of tumor, cyst, lesion, mass, cancer, or any combination thereof in the subject; or (v) any combination of (i)-(iv). Once the set of probabilities is generated, a determination is made (i) that a tumor, cyst, lesion, mass, cancer, or any combination thereof is present in the subject; (ii) of the type or grade of tumor, cyst, lesion, mass, cancer, or any combination thereof present in the subject; (iii) of the classification of the tumor, cyst, lesion, mass, cancer, or any combination thereof in the subject; (iv) of the subtype of tumor, cyst, lesion, mass, cancer, or any combination thereof in the subject; or (v) any combination of (i)-(iv).

In one aspect, the disclosure relates to methods for detecting or determining the presence, type or grade of a tumor in a sample obtained from a subject of interest (e.g., a human). In another aspect, the disclosure relates to determining the presence, type, or grade of a cyst in a sample obtained from a subject of interest (e.g., a human). In yet another aspect, the disclosure relates to determining the presence, type, or grade of lesion in a sample obtained from a subject of interest (e.g., a human). In yet another aspect, the disclosure relates to determining the presence, type, or grade of a mass in a sample obtained from a subject of interest (e.g., a human). In still yet another aspect, the disclosure relates to determining the presence of cancer in a sample obtained from a subject of interest (e.g., a human). For example, in some aspects, the cancer to be determined is a glioma. In another aspect, the disclosure relates to determining the presence of a mass in a subject. In yet another aspect, the disclosure relates to determining the presence of a tumor in a subject. In yet another aspect, the disclosure relates to determining the presence of a cyst in a subject. In still another aspect, the disclosure relates to determining the presence of a lesion in a subject.

In still yet another aspect, the disclosure relates to determining the type cancer in a sample obtained from a subject of interest. For example, in some aspects, the type of cancer that can be determined can be glioma. In still yet another aspect, the disclosure relates to determining the type of tumor in a sample obtained from a subject of interest. In still yet another aspect, the disclosure relates to determining the type of cyst in a sample obtained from a subject of interest. In still yet another aspect, the disclosure relates to determining the type of mass in a sample obtained from a subject of interest. In still yet another aspect, the disclosure relates to determining the type of lesion in a sample obtained from a subject of interest.

In still yet further aspects, the disclosure relates to determining the grade of a cancer in a sample obtained from a subject of interest (e.g., a human). In still yet further aspects, the disclosure relates to determining the grade of a tumor in a sample obtained from a subject of interest (e.g., a human). In still yet further aspects, the disclosure relates to determining the grade of a cyst in a sample obtained from a subject of interest (e.g., a human). In still yet further aspects, the disclosure relates to determining the grade of a mass in a sample obtained from a subject of interest (e.g., a human). In still yet further aspects, the disclosure relates to determining the grade of a lesion in a sample obtained from a subject of interest (e.g., a human).

In still yet another aspect, the present disclosure relates to classifying a cancer in a sample obtained from a subject of interest (e.g., a human). In still yet another aspect, the present disclosure relates to classifying a tumor in a sample obtained from a subject of interest (e.g., a human). In still yet another aspect, the present disclosure relates to classifying a cyst in a sample obtained from a subject of interest (e.g., a human). In still yet another aspect, the present disclosure relates to classifying a mass in a sample obtained from a subject of interest (e.g., a human). In still yet another aspect, the present disclosure relates to classifying a lesion in a sample obtained from a subject of interest (e.g., a human).

In still yet another aspect, the present disclosure relates to subtyping a cancer in a sample obtained from a subject of interest (e.g., a human). In some aspects, the methods involving diagnosing a glioma in a subject of interest. In still yet another aspect, the present disclosure relates to subtyping a tumor in a sample obtained from a subject of interest (e.g., a human). In still yet another aspect, the present disclosure relates to subtyping a cyst in a sample obtained from a subject of interest (e.g., a human). In still yet another aspect, the present disclosure relates to subtyping a mass in a sample obtained from a subject of interest (e.g., a human).

In still yet another aspect, the present disclosure relates to subtyping a lesion in a sample obtained from a subject of interest (e.g., a human).

In other aspects, the disclosure relates to detecting or determining the presence of a pancreatic cyst or pancreatic cyst lesion (PCL) in a subject of interest. In still yet other aspects, disclosure relates to determining the type or grade of pancreatic cyst or PCL. In still other aspects, the disclosure relates to identifying a PCL as a pancreatic adenocarcinoma. In yet another aspect, the disclosure relates to determining the grade and/or classification of a pancreatic cyst or PCL. For example, the methods of the present disclosure can be used to delineate a low grade (e.g., a benign cyst (such as a mucinous cyst)) pancreatic cyst or PCL from a high grade (e.g., a cyst or PCL having malignant potential such as an adenocarcinoma) pancreatic cyst or PCL or high grade dysplasia from invasive adenocarcinoma. A low grade pancreatic cyst or PCL may only require monitoring whereas a high grade pancreatic cyst PCL (e.g., adenocarcinoma) may require surgical intervention.

As mentioned previously, the methods of the present disclosure involve preparing an RNA sequence library. Preparation of the RNA sequence library involves obtaining or isolating extracellular vesicles from a sample obtained from a subject of interest. A subject of interest can be a subject (1) suspected of having a tumor, cyst, lesion, mass, cancer, or any combination thereof; or (2) known to have a tumor, cyst, lesion, mass, cancer, or any combination thereof (such as, for example, for purposes of determining the type of tumor, cyst, lesion, mass, and/or cancer, the grade of the tumor or cancer or the classification or subtype of tumor, cyst, lesion, mass and/or cancer, and/or confirming the presence of the tumor, cyst, lesion, mass, and/or cancer).

The sample used in the methods of the present disclosure can any type of sample obtained from a subject can be used provided that it contains one or more extracellular vesicles, such as, for example, blood, serum, plasma, cyst fluid, (e.g., pancreatic cyst fluid), urine, sputum, saliva, bone marrow, tears, or sweat). In some aspects, the sample is a serum sample. In other aspects, the sample is a plasma sample. In still other aspects, the sample is a cyst fluid sample. The sample can be obtained from a subject using any techniques known in the art. In some aspects, the sample obtained from the subject can be a whole blood sample and serum or plasma obtained from the whole blood sample using routine techniques known in the art such as centrifugation. In other aspects, the serum is a liquid biopsy collected from a resection of a tumor, cyst, lesion, mass, or cancer (e.g., such as a glioma resection). In some aspects, the liquid serum is frozen. In yet further aspects, the amount of frozen serum is at least about 500 microliters. In some aspects, cyst fluid can be obtained using needle aspiration, such as endoscopic ultrasound-guided fine needle aspiration.

Once a sample is obtained from the subject, extracellular vesicles can be isolated using routine techniques known in the art, such as, for example, using centrifugation, ultracentrifugation, magnetic-activated cell sorting size, exclusion chromatography, precipitation, immunoaffinity isolation, or any combination thereof. For example, in some aspects, the EVs can be obtained from frozen serum. When using frozen serum, the extracellular vesicles can be obtained by: (a) thawing the frozen serum (e.g., such as to room temperature); (b) removing residual cells in the thawed serum by centrifugation and retaining the supernatant; (c) incubating the supernatant overnight (the supernatant can be incubated overnight at a temperature of from about 2 to about 8° C., in some aspects, from about 3 to about 5° C., in still further aspects, at about 4° C. (such as, for example, with Invitrogen's total Exosome Isolation Reagent (Invitrogen (Walham, MA) 4478360))); (d) centrifuging the incubated supernatant (e.g., such as, after two days at room temperature) to precipitate the extracellular vesicles (e.g., into a pelI); (e) removing the supernatant; (f) re-suspending the precipitated extracellular vesicles (e.g., pellet) in a buffer (e.g., such as phosphate buffered saline (such as that available from Gibco (Walham, MA) 10010023)); and (g) isolating the extracellular vesicles (e.g., using routine techniques known in the art such as, for example, by using the MagMAX Cell-Free Total Nucleic Acid Isolation Kit (ThermoFisher (Walham, MA) A36716)). In some aspects, the centrifugation in step (b) is performed at about 2000 g for about 30 minutes. In still further aspects, the centrifugation in step (d) is performed at about 10,000 g for about 60 minutes.

Once the EVs are obtained, RNA is obtained or isolated from the EVs. The RNA can be obtained using routine techniques known in the art. For example, the EVs can be digested (e.g., such as with a serine protease) and then lysed (e.g., such as through the use of mechanical force or introduction, hypo/hypertonic solutions, and/or detergent-containing buffers). Once the RNA is obtained, it can be affixed to a solid support (e.g., such as a bead, specifically, a magnetic particle) for library construction. For example, in some aspects, the extraction of RNA from the extracellular vesicles comprises the steps of: (a) digesting the precipitated extracellular vesicles with a serine proteinase (such as Proteinase K) and lysing using routine techniques known in the art; and (b) affixing or attaching the precipitated RNA in extracellular vesicles to a solid support.

One the RNA has been obtained or isolated from the EVs, the RNA sequence library is prepared or constructed using CATS. In some aspects, the CATS library preparation can be modified to utilize (1) polyethylene glycol molecular crowding to increase the efficiency of RNA sequencing; (2) unique molecular identifiers (UMIs), where random base pairs are synthesized on sequence adapters to aid in direct quantification of the RNA template; or (3) combinations of (1) and (2) to allow for direct quantification of each RNA molecule. In still further aspects, the CATS method can be optimized such that single stranded RNA is polyadenylated using a polynucleotide kinase (such as a T4 polynucleotide kinase (such as, for example, NEB M0201S)), dATP, an E. coli Poly (A) polymerase, and a buffer (such as, for example, NEB M0276S) followed by first strand cDNA synthesis in the presence of a poly (dT) anchored oligonucleotide containing a UMI sequence (such as, for example, SMARTscribe Reverse Transcriptase, Takara Bio (San Jose, CA) USA, PN 639538), and 5′-biotin blocked template switch oligonucleotide (TSO), which acts as a second template for the reverse transcriptase, and is included during the first strand synthesis reaction. In some aspects, the first strand synthesis can be followed by digestion with an exonuclease (such as Exonuclease I (available from ThermoFisher)).

Once the RNA sequence library is constructed it can be evaluated and characterized using chip electrophoresis (such as by using Agilent's DNA High Sensitivity Chip (Agilent Technologies Inc. (Santa, Clara, CA)). The RNA sequence library can be sequenced using routine techniques known in the art, such as by next-generation sequencing. For example, the RNA library sequence can be characterized and sequenced using next-generation sequencing systems such as, for example those available from Agilent (e.g., Agilent's 2100 Bioanalyzer System) and Illumina (e.g., Illumina's NovaSeq 6000).

The RNA sequence library prepared from the EVs described above comprises RNA sequences, such as at least full or partial RNA transcripts, retroelements, transposable elements, non-coding RNA, or any combination thereof. In some aspects, the RNA sequences are RNA transcripts. In some aspects, the full or partial RNA transcripts include, but are not limited to, mRNA, miRNA, ncRNA, rRNA, tRNA, snRNA or any combination thereof. In yet other aspects, the RNA transcript can ATRNL1, IL2, or any combination thereof. In yet other aspects, the RNA sequences are retroelements. In some aspects, the retroelements or transposable elements are long interspersed nuclear elements (LINE), short interspersed nuclear elements (SINE), SINE-VNTR-Alu (SVA), long terminal repeat (LTR) retroelements, non-LTR elements, Tyrosine recombinase (YR) retroelements, Penelope like elements (PLEs), pericentromeric satellites, alpha satellites, or any combination thereof. In still another aspect, retroelements such as, long terminal repeat (LTR) retroelements, non-LTR elements, Tyrosine recombinase (YR) retroelements, Penelope retrotransposons (PLEs) or any combination thereof, are highly predictive biomarkers for glioma. In still yet a further aspect, the retroelements, LINE, SINE, Alu, ALR/Alpha or any combination thereof were found to be highly predictive biomarkers for glioma.

As mentioned previously herein, the RNA sequences, such as the full or partial RNA transcripts (e.g., mRNA, miRNA, ncRNA, rRNA, tRNA, snRNA), retroelements, transposable elements, non-coding RNA, or any combination thereof, are next analyzed utilizing a k-mers based machine learning algorithm. Specifically, a processing system is provided which comprises a computer processor and a non-transitory computer memory comprising a database and at least one k-mers based machine learning algorithm. In some aspects, the k-mers based classification algorithm used is iMOKA. More specifically, in this aspect, the RNA sequences (e.g., full or partial RNA transcripts (e.g., mRNA, miRNA, ncRNA, rRNA, tRNA, snRNA), retroelements, transposable elements, non-coding RNA, or any combination thereof) from the RNA library are analyzed using the k-mer based classification algorithm, iMOKA for independent runs of k∈[15,20,25,30,50]. In still another aspect, the iMOKA can be modified to function with custom coding to use multiple length of k. In further aspects, the iMOKA generates k-mer count matrices and prunes uninformative ‘mers’ using a combination of naïve Bayes classification and an entropy filter. In still further aspects, using a combination of naïve Bayes classification and an entropy filter can be used to help reduce the computational burden of rigorously analyzing prohibitively large k-mer matrices.

Once the k-mers based machine learning algorithm completes the analysis of the RNA sequences (e.g., full or partial RNA transcripts (e.g., mRNA, miRNA, ncRNA, rRNA, tRNA, snRNA), retroelements, transposable elements, non-coding RNA, or any combination thereof) the results of k-mers analysis for the subject are generated (“subject k-mers results”). The subject k-mers results are then reported by the processing system. The processing system can supply one or more reference k-mers reference profiles for comparison with the subject k-mers results. More specifically, as discussed previously herein, the one or more reference k-mers profiles are a set of results obtained from one or more suitable control groups (e.g., such as a (i) group of subjects known or determined not to have a tumor, cyst, lesion, mass, and/or cancer; (ii) a group of subjects diagnosed with a tumor, cyst, lesion (e.g., a PCL), mass, and/or cancer; (iii) a group of subjects diagnosed with a specific type of tumor, cyst, lesion (e.g., a low grade or high grade PCL), mass, and/or cancer; (iv) a group of subjects diagnosed with a particular or specific grade of tumor, cyst, lesion, mass, and/or cancer; and/or (v) a group of subjects diagnosed with a specific subtype of tumor, cyst, lesion, mass, and/or cancer), and can be obtained using routine techniques known in the art.

Once the reference-k-mers profile is provided, the k-mers based machine learning algorithm compares the subject k-mers results with those of the reference k-mers profiles to generate a set of probabilities to indicate whether the subject k-mers results are statistically similar to an outcome of interest. This set of probabilities can be communicated (e.g., reported) for further analysis, interpretation, processing and/or display. The result can be communicated (e.g., reported) by the system, such as by a computer, in a document and/or spreadsheet, on a mobile device (e.g., a smart phone), on a website, in an e-mail, or any combination thereof. In some aspects, the set of probabilities are used by a clinician to determine an outcome of interest.

In some aspects, the outcome of interest is to (i) detect and/or identify the presence of a tumor, cyst, lesion, mass, and/or cancer in the subject; (ii) determine the type or grade of tumor, cyst, lesion, mass, or cancer in the subject; (iii) classify the tumor, cyst, lesion, mass, cancer, or any combination thereof in the subject; (iv) determine the subtype of tumor, cyst, lesion, mass, cancer, or any combination thereof in the subject; or (v) any combination of (i)-(iv). Once the set of probabilities is generated, a determination is made using routine techniques known in the art that: (i) a tumor, cyst, lesion, mass, and/or cancer is present (or not present) in the subject; (ii) for subjects that have a tumor, cyst, lesion, mass and/or cancer, the type or grade of tumor, cyst, lesion, mass, and/or cancer present in the subject; (iii) for subjects that have a tumor, cyst, lesion, mass, and/or cancer, the classification of the tumor, cyst, lesion, mass and/or cancer; (iv) for subjects that have a tumor, cyst, lesion, mass, and/or cancer, the subtype of tumor, cyst, lesion, mass, and/or cancer in the subject; or (v) any combination of (i)-(iv).

In some aspects, the reference k-mers profiles described herein are contained in one or more databases (such as a reference k-mers database). In still further aspects, the database is stored on a computational memory chip. In still further aspects, the database is stored on a computer.

### III. Methods for Monitoring the Progression or Recurrence of a Tumor, Cyst, Lesion, Mass, Cancer, or any Combination Thereof in a Subject

In another embodiment, the present disclosure relates to methods for monitoring the progression or recurrence of a tumor, cyst, lesion, mass, cancer, or any combination thereof in a subject of interest using the methods described previously in Section II.

Specifically, the methods comprise preparing, generating, and/or providing a RNA sequence library from RNA isolated from extracellular vesicles in a sample obtained from a subject of interest. The RNA sequence library comprises RNA sequences such as full or partial RNA transcripts (e.g., mRNA, miRNA, ncRNA, rRNA, tRNA, snRNA), retroelements, transposable elements, non-coding RNA, or any combination thereof.

Once the RNA sequence library has been prepared, generated, obtained, and/or provided, a processing system comprising a computer processor and a non-transitory computer memory comprising a database and at least one k-mers based machine learning algorithm is provided to perform the requisite analysis.

In some aspects of the above method, it was discovered that the accuracy of method could be improved by prior to performing or utilizing the k-mers based machine learning algorithm, aligning the RNA sequences in the RNA sequence library with a reference genome sequence using routine techniques known in the art (such as by using a short read aligner such as BowTie, BWA or STAR). These alignments are then collapsed by UMI to accurately quantify the number of unique RNA molecules sequenced.

In some aspects, the RNA sequences (e.g., full or partial RNA transcripts (e.g., mRNA, miRNA, ncRNA, rRNA, tRNA, snRNA), retroelements, transposable elements, non-coding RNA, or any combination thereof), align with the reference genome with at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, or at least 100% sequence identity.

In some aspects, a consensus sequence is generated from the alignment of the RNA sequences with the reference genome, and unique molecular indicators (UMIs). Without wishing to be bound by any theory, the inclusion of this alignment (of the RNA sequences with the reference genome) increases the accuracy of the method by reducing bias from sequencing and/or random error. More specifically, it was found that the use of UMIs in the CATS library preparation could introduce error into the analysis by miscalling polymorphisms in the sequencing due to error. As a result, this created errors in the k-mer analysis that were incorrect due to a single base error. By aligning the RNA sequences from the RNA sequence library (such as full or partial RNA transcripts (e.g., mRNA, miRNA, ncRNA, rRNA, tRNA, snRNA), retroelements, transposable elements, non-coding RNA, or any combination thereof), with a reference genome sequence and utilizing a consensus sequence, the comparability of the k-mers being compared is ensured and the accuracy of the method is increased. Once the alignment is completed, the RNA sequences (e.g., full or partial RNA transcripts (e.g., mRNA, miRNA, ncRNA, rRNA, tRNA, snRNA), retroelements, transposable elements, non-coding RNA, or any combination thereof) can be used in the k-mers based machine learning algorithm.

The k-mers based machine learning algorithm is used to perform the analysis. Specifically, the k-mers based machine learning algorithm used in the method is configured to: (i) apply the machine learning algorithm to the RNA sequence library previously generated (resulting from the alignment with the reference genome) to generate k-mers results for the subject; and (ii) use the subject's k-mers results and a reference k-mers profile to generate a set of probabilities to indicate whether the k-mers results from the subject are statistically similar to an outcome of interest. This set of probabilities can be communicated (e.g., reported) for further analysis, interpretation, processing and/or display. The result can be communicated (e.g., reported) by the system, such as by a computer, in a document and/or spreadsheet, on a mobile device (e.g., a smart phone), on a website, in an e-mail, or any combination thereof. In some aspects, the set of probabilities are used by a clinician to determine an outcome of interest.

In some aspects, the outcome of interest is to identify whether the tumor, cyst, lesion, mass, and/or cancer in the subject has (i) increased or decreased in size; or (ii) has recurred or re-appeared in the subject. Once the set of probabilities is generated, a determination is made whether (i) the tumor, cyst, lesion, mass, and/or cancer in the subject has increased in size and progressed, or, decreased in size and not progressed (e.g., which may indicate the efficacy of the treatment); or (ii) the tumor, cyst, lesion, mass, and/or cancer has reoccurred or re-appeared in the subject.

The one or more reference k-mers profiles used in this method are a set of results obtained from one or more suitable control groups (e.g., such as a (i) group of subjects known not to have a tumor, cyst, lesion, mass, and/or cancer; (ii) a group of subjects diagnosed with a tumor, cyst, lesion, mass, and/or cancer and optionally receiving treatment for the tumor, cyst, lesion, mass, and/or cancer; (iii) a group of subjects diagnosed with a particular type or grade of tumor, cyst, lesion, mass, and/or cancer and optionally receiving treatment for the type or grade of tumor, cyst, lesion, mass, and/or cancer; (iv) a group of subjects diagnosed with a tumor, cyst, lesion, mass, and/or cancer wherein the tumor, cyst, lesion, mass, and/or cancer has increased in size and progressed; (v) a group of subjects diagnosed with a tumor, cyst, lesion, mass, and/or cancer wherein the tumor, cyst, lesion, mass, and/or cancer has decreased in size and not progressed; (vi) a group of subjects previously diagnosed with a tumor, cyst, lesion, mass, and/or cancer wherein the tumor, cyst, lesion, mass, and/or cancer has not reappeared or re-occurred; (vii) a group of subjects previously diagnosed with a tumor, cyst, lesion, mass, and/or cancer wherein the tumor, cyst, lesion, mass, and/or cancer has reappeared or re-occurred; or (viii) any combinations of (i)-(vii)) and can be obtained using routine techniques known in the art.

In some aspects, the subject (e.g., a human) of interest is known or (previously) determined to have a tumor, cyst, lesion, mass, cancer, or any combination thereof, and may optionally be receiving treatment for any said tumor, cyst, lesion, mass, cancer, or any combination thereof. Such treatments will depend on whether the subject has a tumor, cyst, lesion, mass, cancer, or any combination thereof, but will be those typically known in the art, such as surgical treatment (such as, for example, removal or resection of a tumor, cyst, lesion, mass, and/or cancer), chemotherapy, radiation, bone marrow transplant, immunotherapy, hormone therapy, cryoablation, and/or targeted drug therapy (such as, for example, one or more small molecules and/or biologics (such as, for example, an antibody or peptide)). In some aspects, the subject being treated is optionally being monitored. Such monitoring may be to gauge the effectiveness of any treatment. For example, the subject may be monitored to determine whether the size of the tumor, cyst, lesion, mass, and/or cancer has increased (e.g., progressed) or decreased, reoccurred or not reoccurred, or spread to other organs and/or tissues in the subject's body. If it is determined that the treatment is not effective, or that the size of the tumor, cyst, lesion, mass, cancer, or any combination thereof has increased and/or progressed to other locations in the body, the type of treatment may be modified and/or changed. In yet other aspects, the subject of interest may have had a tumor, cyst, lesion, mass, and/or cancer completed treatment and is in remission and being monitored to ensure that the tumor, cyst, lesion, mass, and/or cancer has not re-occurred, re-appeared, or returned.

In some aspects, the subject of interest has been identified or diagnosed as having a pancreatic cyst or PCL. If the subject has been identified or diagnosed as having a low grade or benign pancreatic cyst or PCL, the subject can be monitored for progression of the cyst or PCL to malignant potential (e.g., from a low grade pancreatic cyst or PCL (e.g., benign cyst such as a mucinous cyst) to a high grade pancreatic cyst or PCL (e.g., a cancerous cyst, such as an pancreatic adenocarcinoma).

In further aspects of the above methods can further comprise predicting the survival of the subject based on the determination of whether the tumor, cyst, lesion, mass, cancer, or any combination thereof has or has not progressed in the subject. In some aspects, if the presence of a tumor, cyst, lesion, mass, cancer, or any combination thereof is identified or determined early, it is likely that the likelihood of survival of the subject will increase.

### V. Methods for Improving the Accuracy of Determining Whether a Subject is at Risk of Developing a Cancer or Re-Occurrence or Reappearance of a Cancer

In another embodiment, the present disclosure relates to methods for improving the accuracy of determining whether a subject of interest is at risk of developing a cancer, such as a glioma, or re-occurrence or reappearance of a cancer, such as glioma. Specifically, the methods of the present disclosure comprise (a) preparing, generating, obtaining, and/or providing a RNA sequence library using capture and amplification by tailing and switching (CATS) from RNA isolated from extracellular vesicles obtained from a sample (e.g., serum) of a subject of interest, wherein the RNA sequence library comprises RNA sequences, such as at least one full or partial RNA transcript (e.g., mRNA, miRNA, ncRNA, rRNA, tRNA, snRNA), retroelement, transposable element, non-coding RNA, or any combination thereof, from the RNA isolated from the extracellular vesicles; (b) analyzing the RNA sequences from the RNA sequence library utilizing a k-mers based machine learning algorithm; and (c) determining if the subject is at risk of (i) having or developing a cancer, such as a glioma; or (ii) having the cancer re-occur, reappear or return (e.g., after being treated for said cancer) based on the analysis in step (b). In some aspects, the subject of interest is a subject suspected of having a cancer, such as a glioma. In other aspects, the subject of interest is a subject that had a cancer (e.g., such as a glioma), completed or is completing treatment, and is in remission and being monitored to ensure that the cancer has not re-occurred, re-appeared, or returned.

The methods involve preparing an RNA sequence library. Preparation of the RNA sequence library involves obtaining or isolating extracellular vesicles from a sample obtained from a subject suspected of having a glioma or at high risk of having or developing a glioma. In some aspects, the sample any type of sample obtained from a subject can be used provided that it contains one or more extracellular vesicles, such as, for example, blood, serum, plasma, cyst fluid, (e.g., pancreatic cyst fluid), urine, sputum, saliva, bone marrow, tears, or sweat. In some aspects, the sample is a serum sample. In other aspects, the sample is a plasma sample. The serum or plasma sample can be obtained from a subject using any techniques known in the art. In some aspects, the sample obtained from the subject can be a whole blood sample and serum or plasma obtained from the whole blood sample using routine techniques known in the art such as centrifugation. In other aspects, the serum is a liquid biopsy collected from a resection of a tumor, cyst, lesion, mass, or cancer (e.g., such as a glioma resection). In some aspects, the liquid serum is frozen. In yet further aspects, the amount of frozen serum is at least about 500 microliters.

Once a sample is obtained from the subject, extracellular vesicles can be isolated using routine techniques known in the art, such as, for example, using centrifugation, ultracentrifugation, magnetic-activated cell sorting size, exclusion chromatography, precipitation, immunoaffinity isolation, or any combination thereof. For example, in some aspects, the EVs can be obtained from frozen serum. When using frozen serum, the extracellular vesicles can be obtained by: (a) thawing the frozen serum (e.g., such as to room temperature); (b) removing residual cells in the thawed serum by centrifugation and retaining the supernatant; (c) incubating the supernatant overnight (the supernatant can be incubated overnight at a temperature of from about 2 to about 8° C., in some aspects, from about 3 to about 5° C., in still further aspects, at about 4° C. (such as, for example, with Invitrogen's total Exosome Isolation Reagent (Invitrogen (Walham, MA) 4478360))); (d) centrifuging the incubated supernatant (e.g., such as, after two days at room temperature) to precipitate the extracellular vesicles (e.g., into a pellet); (e) removing the supernatant; (f) re-suspending the precipitated extracellular vesicles (e.g., pellet) in a buffer (e.g., such as phosphate buffered saline (such as that available from Gibco (Walham, MA) 10010023)); and (g) isolating the extracellular vesicles (e.g., using routine techniques known in the art such as, for example, by using the MagMAX Cell-Free Total Nucleic Acid Isolation Kit (ThermoFisher (Walham, MA) A36716)). In some aspects, the centrifugation in step (b) is performed at about 2000 g for about 30 minutes. In still further aspects, the centrifugation in step (d) is performed at about 10,000 g for about 60 minutes.

Once the EVs are obtained, RNA is obtained or isolated from the EVs. The RNA can be obtained using routine techniques known in the art. For example, the EVs can be digested (e.g., such as with a serine protease) and then lysed (e.g., such as through the use of mechanical force or introduction, hypo/hypertonic solutions, and/or detergent-containing buffers). Once the RNA is obtained, it can be affixed to a solid support (e.g., such as a bead, specifically, a magnetic particle) for library construction. For example, in some aspects, the extraction of RNA from the extracellular vesicles comprises the steps of: (a) digesting the precipitated extracellular vesicles with a serine proteinase (such as Proteinase K) and lysing using routine techniques known in the art; and (b) affixing or attaching the precipitated RNA in extracellular vesicles to a solid support.

One the RNA has been obtained or isolated from the EVs, the RNA sequence library is prepared or constructed using CATS. In some aspects, the CATS library preparation can be modified to utilize (1) polyethylene glycol molecular crowding to increase the efficiency of RNA sequencing; (2) unique molecular identifiers (UMIs), where random base pairs are synthesized on sequence adapters to aid in direct quantification of the RNA template; or (3) combinations of (1) and (2) to allow for direct quantification of each RNA molecule. In still further aspects, the CATS method can be optimized such that single stranded RNA is polyadenylated using a polynucleotide kinase (such as a T4 polynucleotide kinase (such as, for example, NEB M0201S)), dATP, an E. coli Poly (A) polymerase, and a buffer (such as, for example, NEB M0276S) followed by first strand cDNA synthesis in the presence of a poly (dT) anchored oligonucleotide containing a UMI sequence (such as, for example, SMARTscribe Reverse Transcriptase, Takara Bio (San Jose, CA) USA, PN 639538), and 5′-biotin blocked template switch oligonucleotide (TSO), which acts as a second template for the reverse transcriptase, and is included during the first strand synthesis reaction. In some aspects, the first strand synthesis can be followed by digestion with an exonuclease (such as Exonuclease I (available from ThermoFisher)).

Once the RNA sequence library is constructed it can be evaluated and characterized using chip electrophoresis (such as by using Agilent's DNA High Sensitivity Chip (Agilent Technologies Inc. (Santa, Clara, CA)). The RNA sequence library can be sequenced using routine techniques known in the art, such as by next-generation sequencing. For example, the RNA library sequence can be characterized and sequenced using next-generation sequencing systems such as, for example those available from Agilent (e.g., Agilent's 2100 Bioanalyzer System) and Illumina (e.g., Illumina's NovaSeq 6000).

The RNA sequence library prepared from the EVs described above comprises RNA sequences, such as one or more full or partial RNA transcripts (e.g., mRNA, miRNA, ncRNA, rRNA, tRNA, snRNA), retroelements, transposable elements, non-coding RNA, or any combination thereof. In some aspects, the retroelements are long terminal repeat (LTR) retroelements, non-LTR elements, Tyrosine recombinase (YR) retroelements, Penelope retrotransposons (PLEs) or any combination thereof. In still another aspect, retroelements such as, long terminal repeat (LTR) retroelements, non-LTR elements, Tyrosine recombinase (YR) retroelements, Penelope retrotransposons (PLEs) or any combination thereof, are highly predictive biomarkers for glioma. In still yet a further aspect, the retroelements, LINE, SINE, Alu, ALR/Alpha or any combination thereof were found to be highly predictive biomarkers for glioma.

In some aspects of the above method, it was discovered that the accuracy of method could be improved by aligning the RNA sequences in the RNA sequence library with a reference genome sequence using routine techniques known in the art (such as by using a short read aligner such as BowTie, BWA or STAR). These alignments are then collapsed by UMI to accurately quantify the number of unique RNA molecules sequences. In some aspects, when determining whether a subject has or is at risk of a glioma, reference genomes such as hg38 or hg19 can be used.

In some aspects, the RNA sequences (e.g., full or partial RNA transcripts (e.g., mRNA, miRNA, ncRNA, rRNA, tRNA, snRNA), retroelements, transposable elements, non-coding RNA, or any combination thereof), align with the reference genome with at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, or at least 100% sequence identity.

In some aspects, a consensus sequence is generated from the alignment of the RNA sequences with the reference genome, and unique molecular indicators (UMIs). Without wishing to be bound by any theory, the inclusion of this alignment (of the RNA sequences with the reference genome) increases the accuracy of the method by reducing bias from sequencing and/or random error. More specifically, it was found that the use of UMIs in the CATS library preparation could introduce error into the analysis by miscalling polymorphisms in the sequencing due to error. As a result, this created errors in the k-mer analysis that were incorrect due to a single base error. By aligning the RNA sequences from the RNA sequence library (such as the full or partial RNA transcripts (e.g., mRNA, miRNA, ncRNA, rRNA, tRNA, snRNA), retroelements, transposable elements, non-coding RNA, or any combination thereof), with a reference genome sequence and utilizing a consensus sequence, the comparability of the k-mers being compared is ensured and the accuracy of the method is increased.

Once the alignment is completed, the RNA sequences (e.g., full or partial RNA transcripts (e.g., mRNA, miRNA, ncRNA, rRNA, tRNA, snRNA), retroelements, transposable elements, non-coding RNA, or any combination thereof) are analyzed utilizing a k-mers based machine learning algorithm. Specifically, a processing system is provided which comprises a computer processor and a non-transitory computer memory comprising a database and at least one k-mers based machine learning algorithm. In some aspects, the k-mers based classification algorithm used is iMOKA. More specifically, in this aspect, the aligned RNA sequences from the RNA library are analyzed using the k-mer based classification algorithm, iMOKA for independent runs of k∈[15,20,25,30,50]. In still another aspect, the iMOKA can be modified to function with custom coding to use multiple length of k. In further aspects, the iMOKA generates k-mer count matrices and prunes uninformative ‘mers’ using a combination of naïve Bayes classification and an entropy filter. In still further aspects, using a combination of naïve Bayes classification and an entropy filter can be used to help reduce the computational burden of rigorously analyzing prohibitively large k-mer matrices.

As mentioned above, once aligned, the RNA sequences (e.g., full or partial RNA transcripts (e.g., mRNA, miRNA, ncRNA, rRNA, tRNA, snRNA), retroelements, transposable elements, non-coding RNA, or any combination thereof) are analyzed utilizing a k-mers based machine learning algorithm. Specifically, the k-mers based machine learning algorithm is configured to first apply the machine learning algorithm to the aligned RNA sequences (full or partial RNA transcripts (e.g., mRNA, miRNA, ncRNA, rRNA, tRNA, snRNA), retroelements, transposable elements, non-coding RNA, or any combination thereof) to generate or produce k-mers results for the subject of interest (“subject k-mers results”). Once the subject k-mers results are generated, these results are obtained and reported by the algorithm.

The processing system can supply one or more reference k-mers reference profiles for comparison with the subject k-mers results. More specifically, the one or more reference k-mers profiles are a set of results obtained from one or more suitable control groups (e.g., such as a (i) group of subjects known or determined not to have cancer, such as a glioma; (ii) a group of subjects diagnosed with a cancer, such a glioma; (iii) a group of subjects previously diagnosed with a cancer wherein the cancer has not reappeared or re-occurred; and/or (vi) a group of subjects previously diagnosed with a cancer, wherein the cancer has reappeared or re-occurred); and can be obtained using routine techniques known in the art.

Once the reference k-mers profile is provided, the k-mers based machine learning algorithm compares the subject k-mers results with those of the reference k-mers profile to generate a set of probabilities to indicate whether the subject k-mers results are statistically similar to an outcome of interest. This set of probabilities can be communicated (e.g., reported) for further analysis, interpretation, processing and/or display. The result can be communicated (e.g., reported) by the system, such as by a computer, in a document and/or spreadsheet, on a mobile device (e.g., a smart phone), on a website, in an e-mail, or any combination thereof. In some aspects, the set of probabilities are used by a clinician to determine an outcome of interest.

In some aspects, the outcome of interest is to identify the risk of cancer in the subject or re-occurrence, reappearance or return of cancer in a subject. For example, in some aspects, the outcome of interest is to identify the risk of a glioma in a subject or re-occurrence or reappearance of a glioma in a subject.

In some aspects, the reference k-mers profiles described herein are contained in one or more databases (such as a reference k-mers database). In still further aspects, the database is stored on a computational memory chip. In still further aspects, the database is stored on a computer.

VI. Systems for (i) Detecting or Determining the Presence, Type, Grade, or Classification of a Tumor, Cyst, Lesion, Mass, and/or Cancer, or (ii) Classifying or Subtyping a Tumor, Cyst, Lesion, Mass, and/or Cancer in a Subject

In another embodiment, the present disclosure relates to a system for (i) detecting determining the presence, type, grade or classification of a tumor, cyst, lesion, mass, cancer, or any combination thereof in a sample obtained from a subject; or (ii) classifying or subtyping a tumor, cyst, lesion, mass, cancer, or any combination thereof in sample obtained from a subject. In this aspect, the system comprises (a) an RNA sequence library generated, prepared and/or obtained using capture and amplification by tailing and switching (CATS) from RNA isolated from extracellular vesicles from a sample from a subject having or suspected having a tumor, cyst, lesion, mass, cancer, or any combination thereof, wherein the RNA sequence library comprises RNA sequences, such as atone or more full or partial RNA transcripts (e.g., mRNA, miRNA, ncRNA, rRNA, tRNA, snRNA), retroelements, transposable elements, non-coding RNA, or any combination thereof, from the RNA isolated from the extracellular vesicles; (b) a k-mers based machine learning algorithm for analyzing the RNA sequences (e.g., full or partial RNA transcripts (e.g., mRNA, miRNA, ncRNA, rRNA, rRNA, snRNA), retroelements, transposable elements, non-coding RNA, or any combination thereof) from the RNA sequence library; and (c) a reference database for determining the presence, type, grade or classification of the tumor, cyst, lesion, mass, cancer, or any combination thereof, or subtyping the tumor, cyst, lesion, mass, cancer, or any combination thereof in the sample based the analysis performed in step (b). More specifically, the RNA sequences (e.g., full or partial RNA transcripts (e.g., mRNA, miRNA, ncRNA, rRNA, tRNA, snRNA), retroelements, transposable elements, non-coding RNA, or any combination thereof) from the RNA sequence library analyzed in step (b) can be compared with the reference database to (i) determine the presence, type or grade of a tumor, cyst, lesion, mass, cancer, or any combination thereof in a sample obtained from a subject; or (ii) subtype a cancer in sample obtained from a subject.

In the above system, the sample obtained from the subject can be any type of sample provided that it contains one or more extracellular vesicles, such as, for example, blood, serum, plasma, cyst fluid, (e.g., pancreatic cyst fluid), urine, sputum, saliva, bone marrow, tears, or sweat. In some aspects, the sample is a serum sample. In some aspects, the sample is a blood sample. In some aspects, the sample is a plasma sample. In other aspects, the sample is a cyst fluid sample.

In the above system, the RNA sequence library can be prepared as described in Section II. Additionally, the k-mers based machine algorithm and analysis can be performed as described as also described in Section II. Additionally, in some aspects, the system can further include an instrument for performing the k-mers based machine learning algorithm. An example of such an instrument is a computer or processing system.

In some aspects, the system relates to determining the presence of a tumor, cyst, lesion, mass, cancer, or any combination thereof in a subject of interest. In another aspect, the system relates to determining the type of tumor, cyst, lesion, mass, cancer, or any combination thereof in a subject of interest. In still other aspects, the system relates to determining the grade of a tumor, cyst, lesion, mass, cancer, or any combination thereof in a subject of interest. In still other aspects, the system relates to classifying a tumor, cyst, lesion, mass, cancer, or any combination thereof in a subject of interest. In still further aspects, the system relates to subtyping a cancer in a subject of interest. In some aspects, the subject is a human. In some aspects, a “subject of interest” refers to a subject that has or is suspected of having a tumor, cyst, lesion, mass, cancer, or any combination thereof.

In another aspect, the system can contain a reference database for (1) detecting or determining the presence, type, or grade of a tumor, cyst, lesion, mass, cancer, or any combination thereof in a subject; or (2) classifying or subtyping a tumor, cyst, lesion, mass, cancer, or any combination thereof in a sample obtained from a subject. The reference database can contain one or more reference k-mers profiles for use in performing the analysis. The reference k-mers profiles are a set of results obtained from one or more suitable control groups (e.g., such as a (i) group of subjects known or determined not to have a tumor, cyst, lesion, mass, and/or cancer; (ii) a group of subjects diagnosed with a tumor, cyst, lesion (e.g., a PCL), mass, and/or cancer; (iii) a group of subjects diagnosed with a specific type of tumor, cyst, lesion (e.g., a low grade or high grade PCL), mass, and/or cancer; (iv) a group of subjects diagnosed with a particular or specific grade of tumor, cyst, lesion, mass, and/or cancer; and/or (v) a group of subjects diagnosed with a specific subtype of tumor, cyst, lesion, mass, and/or cancer), and can be obtained using routine techniques known in the art.

Once the reference database supplies the reference k-mers profiles, the k-mers based machine learning algorithm compares the subject k-mers results with those of the reference k-mers profiles to generate a set of probabilities to indicate whether the subject k-mers results are statistically similar to an outcome of interest. These set of probabilities can be communicated (e.g., reported) for further analysis, interpretation, processing and/or display. The result can be communicated (e.g., reported) by the system, such as by a computer, in a document and/or spreadsheet, on a mobile device (e.g., a smart phone), on a website, in an e-mail, or any combination thereof. In some aspects, the set of probabilities are used by a clinician to determine an outcome of interest.

In some aspects, the outcome of interest is to (i) detect and/or identify the presence of a tumor, cyst, lesion, mass, and/or cancer in the subject; (ii) determine the type or grade of tumor, cyst, lesion, mass, or cancer in the subject; (iii) classify the tumor, cyst, lesion, mass, cancer, or any combination thereof in the subject; (iv) determine the subtype of tumor, cyst, lesion, mass, cancer, or any combination thereof in the subject; or (v) any combination of (i)-(iv). Once the set of probabilities is generated, a determination is made using routine techniques known in the art that: (i) a tumor, cyst, lesion, mass, and/or cancer is present (or not present) in the subject; (ii) for subjects that have a tumor, cyst, lesion, mass and/or cancer, the type or grade of tumor, cyst, lesion, mass, and/or cancer present in the subject; (iii) for subjects that have a tumor, cyst, lesion, mass, and/or cancer, the classification of the tumor, cyst, lesion, mass and/or cancer; (iv) for subjects that have a tumor, cyst, lesion, mass, and/or cancer, the subtype of tumor, cyst, lesion, mass, and/or cancer in the subject; or (v) any combination of (i)-(iv).

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described. All publications mentioned herein are incorporated herein by reference to disclose and described the methods and/or materials in connection with which the publications are cited.

The publications discussed herein are provided solely for their disclosure prior to the filing date of the present application and each is incorporated by reference in its entirety. Nothing herein is to be construed as an admission that the present disclosure is not entitled to antedate such publication by virtue of prior disclosure. Further, the dates of publication provided may be different from the actual publication dates which may need to be independently confirmed.

### EXAMPLES

### Example 1: Glioma Extracellular Vesical Based Liquid Biopsy-GlioEV

A novel and comprehensive platform was developed for the isolation, sequencing, and quantification of diverse RNA species inside of extracellular vesicles (EVs) fine-tuned for glioma prediction on archived samples. Our approach is informed by broad experience isolating, sequencing and categorizing viruses, which are morphologically similar to EVs. 3 EV isolation techniques and 6 RNA extraction approaches were tested, then evaluated each approach with including size and concentration measurement using bioanalyzer, qPCR, and sequencing, with reference based and agnostic bioinformatic analysis. From that process. a robust approach was developed that maintains a high yield of diverse RNA in EVs in fresh and archived samples. The results of this long iterative process form the basis of the EV isolation and RNA extraction for GlioEV.

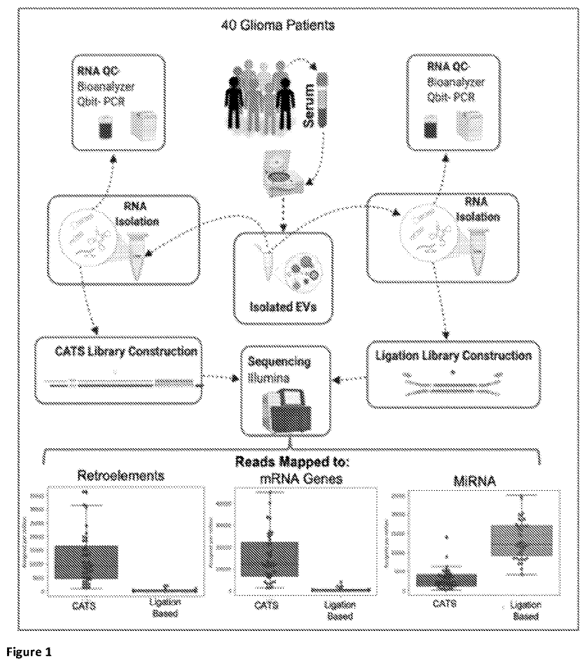

Analysis on 40 UCSF glioma patients (10 Oligodendroglioma, 10 Astrocytoma, 10 Grade IV Astrocytoma (formerly IDHmt GBM), 10 Glioblastoma) was conducted on serum collected at primary resection and stored at −80° C. for 3 years (the maximum time selected for archived glioma samples). The entire EV isolation and RNA extraction process was conducted in parallel using a traditional ligation-based RNA library preparation (Lexogen) and with our custom Capture by Amplification and Tail Switching (CATS) based library preparation on the same subjects serum (FIG. 1). This was done as a final step in the refinement of GlioEV to choose the library preparation approach that yields the most informative RNA features for glioma subtype prediction.

A CATS library preparation was modified to function with extremely low RNA input by utilizing polyethylene glycol crowding, custom oligo alterations to increase template switching efficiency, and unique molecular identifiers (UMI) that allow for direct quantification of each RNA molecule. Comparison of ligation-based library preparation to our CATS preparation has revealed substantial differences in the types of RNA sequenced. This CATS protocol sequences a significantly greater number of RNA transcripts and retroelements inside of EVs (FIG. 1). However, proportionately, CATS sequences less miRNA than the ligation-based approach, yet a relatively high concordance of unique miRNA detected was observed. Several other studies have shown that this bias in ligation approaches is in part due to 2′-O-methyl modifications on the 3′ terminal nucleotide ligation efficacy.²⁰

Using traditional reference based differential expression analysis of EVs have cataloged miRNA species and gene transcripts previously associated with glioma, such as miR-92b, miR-182, miR-221 and miR-340.²¹⁻²³ Associations with tumor grade for several RNA transcripts, including ATRNL1 and IL2, which have been previously linked to glioma^{22,24} were also detected. Furthermore, for the first time, retroelements in EVs were categorized that are strongly associated with glioma subtypes, with corresponding differential expression of these retroelements in tumor/normal RNA-seq and glioma subtype in the TCGA²⁵. This finding is a critical innovation made possible by the CATS library preparation. Retroelement reactivation could be described as a hallmark of cancer, yet the significant functional relevance of these genetic elements that make up the majority of the human genome is just beginning to be understood²⁶. What is clear is that in a dysregulated cancer cell, retroelements that are usually silenced in healthy cells, are overexpressed.

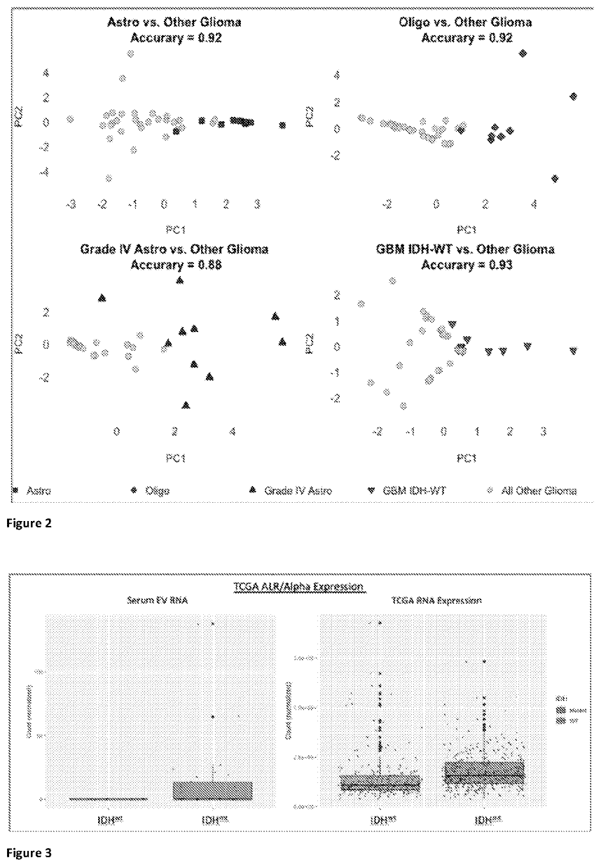

The final and key piece of methodological innovation is the utilization of an agnostic k-mer based machine learning algorithm to predict glioma subtype²⁷. This approach creates a k-mer matrix for iterative feature selection with internal cross validation, followed by a random forest optimization of subtype classification. This approach predicted glioma subtype with an accuracy (the average of 10-fold, leave one out, cross validated model fitting) of 88-93% using only features inside of serum-derived EVs, illustrating the potential of this tool to greatly improve tumor detection and classification (FIG. 2). In contrast, the more traditional ligation-based library preparation, applied to the same subjects using the same analysis platform, achieved a maximum accuracy of 37% for subtype classification. This is because the ligation-based library fails to sequence many of the most informative features, which are generally not miRNA, but retroelements and other RNA isoforms. This key observation is the result of our development and refinement of the GlioEV platform by quantitively analyzing multiple methods fine-tuned to specifically to predict glioma subtypes with no a priori assumptions.

These results are the first that demonstrate the utility of retroelementsin the context of a liquid biopsy. Many of the retroelements, including LINE, Alu, ALR/Alpha, and LTR elements, that were observed as highly predictive in GlioEV, follow a similar expression pattern in RNA-seq from glioma tumors. For example, using this data, a GlioEV classifier was trained to identify IDH-mutated (IDHmt) tumors, where 93% accuracy was achieved, in which the most predictive feature selected by machine learning was a retroelement. This retroelement, broadly defined as the satellite ALR/Alpha that exists near the centromere and pericentromere was present almost exclusively in the EVs from patients' serum with IDHmt tumors. This same pattern is clear in The Cancer Genome Atlas (TCGA) gliomas, where the satellite ALR/Alpha is significantly up regulated in RNA-seq from IDHmt tumors (FIG. 3). This suggests that these retroelements features are packaged inside of EVs in the tumor, cross the blood brain barrier, and are detectable in serum.

### GlioEV—Isolation and Sequencing

Briefly, serum EVs are isolated and RNA extracted, sequencing libraries are prepared using an in house CATS based protocol including unique molecular identifiers (UMIs) that have been shown herein to be superior for glioma prediction ligation. Precisely, 500 microliters frozen serum is slowly thawed to room temperature followed by centrifugation at 2000 g for 30 minutes to remove any residual cells. The supernatant is then incubated overnight at 4° C. with Invitrogen's Total Exosome Isolation Reagent (Invitrogen 4478360). On day two, the sample is centrifuged at 10,000×g for 60 minutes at room temperature to precipitate the EVs. The supernatant is removed and discarded. The pellet, which contains the EVs, is re-suspended in 1 mL phosphate buffered saline (Gibco 10010023) prior to extraction using the MagMAX Cell-Free Total Nucleic Acid Isolation Kit (ThermoFisher A36716). Precipitated EVs are digested with Proteinase K and lysed according to the manufacturer's protocol. EV RNA is then bound to magnetic beads, which are washed prior to concentration and elution of the RNA. Following the principals for ligation free library preparation using Capture and Amplification by Tailing and Switching (CATS) originally laid out by Turchinovich et al.²⁹, and further optimizedas described herein, single stranded RNA is poly adenylated using T4 polynucleotide Kinase (NEB M0201S), dATP and E. coli Poly (A) polymerase, and buffer (NEB M0276S) followed by first strand cDNA synthesis in the presence of a poly (dT) anchored oligonucleotide containing a UMI sequence (SMARTscribe Reverse Transcriptase, Takara Bio USA, PN 639538). 5′biotin blocked template switching oligo, acting as a second template for the reverse transcriptase, is further included during the first strand synthesis reaction. First strand synthesis is followed by digestion with Exonuclease I (Thermo, PN FEREN0581), to remove single stranded templates. Second strand synthesis with unique dual index primers compatible to the Illumina Next Generation Sequencing platform is performed for 25 cycles (Terra PCR Direct Polymerase, Takara Bio USA, PN 639270), followed by library clean up with AMPure XP SPRI Beads (Beckman Coulter, A63881). Libraries are characterized using Agilent's DNA High Sensitivity Chip (Agilent Technologies Inc, PN 5067-4626), prior to equimolar multiplexing and sequencing on Illumina's NovaSeq 6000.

### GlioEV—Bioinformatics

Raw sequencing data is downloaded from the QB3 sequencing core where initial Illumina QC is performed. Additional QC with BBTools' BBDuk2 (Lawrence Berkeley National Lab) was conducted in accordance with accepted standards for basic sequencing QC such as adapter trimming, quality trimming, GC content, etc. However, filtering by read length less than 10 bp was performed to ensure miRNA are analyzed and that the totality of RNA/DNA size range is captured for downstream analyses of fragment lengths. Further, although many of the RNA species and some DNA species shorter than 150 bp, 150 bp PE sequencing was utilized to capture complete fragment lengths. The aligners Bowtie2³⁰, STAR³¹, Kallisto³² and Diamond³³ were used to align QC′d sequencing reads to the human genome (hg38), transcriptome, miRBase's miRNA reference and viral and bacterial references for downstream analysis. Every read was identified. Alignments from STAR are analyzed to produce count matrices using FeatureCounts which are then be analyzed using DESeq2³⁴ and those from Kallisto for differential expression. Reads are analyzed for Repetitive and Transposable Element content with REdiscoverTE²⁵, any circular RNA with CIRCexplorer2.³⁵

### GlioEV—Statistical Analysis Summary

A 70/30 training/test split of the data with identically sized held-out subgroups was used to ensure metrics of performance of the model are validated on an independent set. Simultaneously, model prediction for major glioma subtype was run based on prognostically significant somatic mutations as described in WHO 2021. To select the best set of EV RNA-seq based features for classification, traditional differential expression analysis was explored, and reference free k-mer based methods. Each approach relies on the use of random forest (RF) classifiers using the provided features to construct a final classifier model. The RF algorithm is a supervised machine learning method for learning patterns in data which generalize well and makes predictions by aggregating information learned from thousands of random decision trees using a majority-rule voting scheme.

### GlioEV—Traditional Differential Expression Analysis

Aligned/pseuo-aligned EV RNA-seq data is analyzed using DESeq2/Sleuth and validated using an independent differential expression (DE) software, EdgeR. To reduce the burden of multiple testing, prior to DE analysis, only elements which have non-zero values in >30% of samples in at least one subgroup. Each DE model is adjusted for age at blood draw, sex, and reported ancestry. The analysis of RNA species follows the same DE pipeline. Significant DE elements between subgroups are defined as those with a Benjamini-Hochberg corrected p-value p<0.05 (two-sided test) and a >1.75-fold change in expression. The collection of RNA differentially expressed elements are pruned for independence (pairwise correlation r²<0.4), where the element with lowest DE p-value in each pairwise comparison are retained. An RF is be trained on the same samples using the resulting elements as features. Out-of-box (OOB) score, a metric unique to RFs, which measures predicted performance on unseen data, are used to tune hyperparameters. The RF with the highest OOB score using the training dataset is nominated as the best DE-based classifier.

### GlioEV—K-Mer Based Prediction/Classifiers

K-mer based approaches have been shown to discover novel genetic associations by avoiding the bias/data loss possible from long bioinformatic pipelines. EV RNA-seq data is analyzed using the k-mer based classification algorithm, iMOKA²⁷, for independent runs of k∈[15,20,25,30,50]. iMOKA generates k-mer count matrices and prunes uninformative ‘mers’ using a combination of naïve Bayes classification and an entropy filter, both of which help reduce the computational burden of rigorously analyzing prohibitively large k-mer matrices. The algorithm keeps mers which individually have some classification ability (cross-validated average accuracy >65%), removes correlated features, and uses the resulting mers to construct a RF classification model. The iMOKAs functionality has been extended with custom coding to use multiple lengths of k to ensure the most predictive length k is utilized. Across the various RFs constructed, one for each value of k, the best mer-based classifier are the ‘k’ which achieves the highest OOB score from the training dataset.

### Example 2: Pancreatic Cysts

The incidence of pancreatic cystic lesions (PCL) continues to increase, PCL are incidentally detected in more than a million patients annually in the U.S. and represent an opportunity for early detection of pancreatic adenocarcinoma, the third leading cause of cancer-related deaths in the United States. The accurate identification of cysts with high grade dysplasia or invasive adenocarcinoma, together referred to as (HGD/AN), that warrant surgical intervention represents a critical unmet need in the management of patients with PCLs. Fear of pancreatic cancer has led to overdiagnosis and unnecessary resection of benign PCLs with substantial attendant mortality and morbidity. It is also clear that early detection of lesions with HGD is a priority because robust studies show that once a mucinous cyst develops an invasive component, the likelihood of long-term survival is significantly decreased. Given the relatively low incidence of pancreatic adenocarcinoma among patients with pancreatic cysts, especially relative to the high overall cyst prevalence, most pancreatic cysts never develop invasive cancer. Thus, accurate classification and less invasive monitoring of pancreatic cysts and their malignant potential remains a critical unmet need.

### Materials and Methods

The extracellular vesicle (EV) sequencing and analysis approach described in Example 1 was applied to pancreatic cyst fluid to assess the ability to risk stratify pancreatic cysts. Extracellular vesicles were isolated and sequenced RNA extracted from cyst fluid from 10 patients with mucinous cysts with confirmed histology (4 low grade dysplasia (LGD), 2 high grade dysplasia (HGD), and 4 adenocarcinoma (AN) per UCSF Pathology review).

Briefly, cyst fluid EVs are isolated and RNA extracted, sequencing libraries are prepared using an in-house CATS based protocol including unique molecular identifiers (UMIs) that have been shown herein to be superior for glioma prediction ligation. Precisely, 500 microliters frozen cyst fluid is slowly thawed to room temperature followed by centrifugation at 2000 g for 30 minutes to remove any residual cells. The supernatant is then incubated overnight at 4° C. with Invitrogen's Total Exosome Isolation Reagent (Invitrogen 4478360). On day two, the sample was centrifuged at 10,000×g for 60 minutes at room temperature to precipitate the EVs. The supernatant is removed and discarded. The pellet, which contains the EVs, is re-suspended in 1 mL phosphate buffered saline (Gibco 10010023) prior to extraction using the MagMAX Cell-Free Total Nucleic Acid Isolation Kit (ThermoFisher A36716). Precipitated EVs are digested with Proteinase K and lysed according to the manufacturer's protocol. EV RNA is then bound to magnetic beads, which are washed prior to concentration and elution of the RNA. Following the principals for ligation free library preparation using Capture and Amplification by Tailing and Switching (CATS) originally laid out by Turchinovich et al.²⁹, and further optimized as described herein, single stranded RNA is poly adenylated using T4 polynucleotide Kinase (NEB M0201S), dATP and E. coli Poly (A) polymerase, and buffer (NEB M0276S) followed by first strand cDNA synthesis in the presence of a poly (dT) anchored oligonucleotide containing a UMI sequence (SMARTscribe Reverse Transcriptase, Takara Bio USA, PN 639538). 5′biotin blocked template switching oligo, acting as a second template for the reverse transcriptase, is further included during the first strand synthesis reaction. First strand synthesis is followed by digestion with Exonuclease I (Thermo, PN FEREN0581), to remove single stranded templates. Second strand synthesis with unique dual index primers compatible to the Illumina Next Generation Sequencing platform is performed for 25 cycles (Terra PCR Direct Polymerase, Takara Bio USA, PN 639270), followed by library clean up with AMPure XP SPRI Beads (Beckman Coulter, A63881). Libraries are characterized using Agilent's DNA High Sensitivity Chip (Agilent Technologies Inc, PN 5067-4626), prior to equimolar multiplexing and sequencing on Illumina's NovaSeq 6000.

Raw sequencing data is downloaded from the QB3 sequencing core where initial Illumina QC is performed. Additional QC with BBTools' BBDuk2 (Lawrence Berkeley National Lab) was conducted in accordance with accepted standards for basic sequencing QC such as adapter trimming, quality trimming, GC content, etc. However, filtering by read length less than 10 bp was performed to ensure miRNA are analyzed and that the totality of RNA/DNA size range is captured for downstream analyses of fragment lengths. Further, although many of the RNA species and some DNA species shorter than 150 bp, 150 bp PE sequencing was utilized to capture complete fragment lengths. The aligners Bowtie2³⁰, STAR³¹, Kallisto³² and Diamond³³ were used to align QC′d sequencing reads to the human genome (hg38), transcriptome, miRBase's miRNA reference and viral and bacterial references for downstream analysis. Every read was identified. Alignments from STAR are analyzed to produce count matrices using FeatureCounts which are then be analyzed using DESeq2³⁴ and those from Kallisto for differential expression. Reads are analyzed for Repetitive and Transposable Element content with REdiscoverTE²⁵, any circular RNA with CIRCexplorer2.³⁵

Aligned/pseuo-aligned EV RNA-seq data is analyzed using DESeq2/Sleuth and validated using an independent differential expression (DE) software, EdgeR. To reduce the burden of multiple testing, prior to DE analysis, only elements which have non-zero values in >30% of samples in at least one subgroup. Each DE model is adjusted for age at blood draw, sex, and reported ancestry. The analysis of RNA species follows the same DE pipeline. Significant DE elements between subgroups are defined as those with a Benjamini-Hochberg corrected p-value p<0.05 (two-sided test) and a >1.75-fold change in expression. The collection of RNA differentially expressed elements are pruned for independence (pairwise correlation r²<0.4), where the element with lowest DE p-value in each pairwise comparison are retained. An RF is be trained on the same samples using the resulting elements as features. Out-of-box (OOB) score, a metric unique to RFs, which measures predicted performance on unseen data, are used to tune hyperparameters. The RF with the highest OOB score using the training dataset is nominated as the best DE-based classifier.

K-mer based approaches have been shown to discover novel genetic associations by avoiding the bias/data loss possible from long bioinformatic pipelines. EV RNA-seq data is analyzed using the k-mer based classification algorithm, iMOKA²⁷, for independent runs of k∈[15,20,25,30,50]. iMOKA generates k-mer count matrices and prunes uninformative ‘mers’ using a combination of naïve Bayes classification and an entropy filter, both of which help reduce the computational burden of rigorously analyzing prohibitively large k-mer matrices. The algorithm keeps mers which individually have some classification ability (cross-validated average accuracy >65%), removes correlated features, and uses the resulting mers to construct a RF classification model. The iMOKAs functionality has been extended with custom coding to use multiple lengths of k to ensure the most predictive length k is utilized. Across the various RFs constructed, one for each value of k, the best mer-based classifier are the ‘k’ which achieves the highest OOB score from the training dataset.

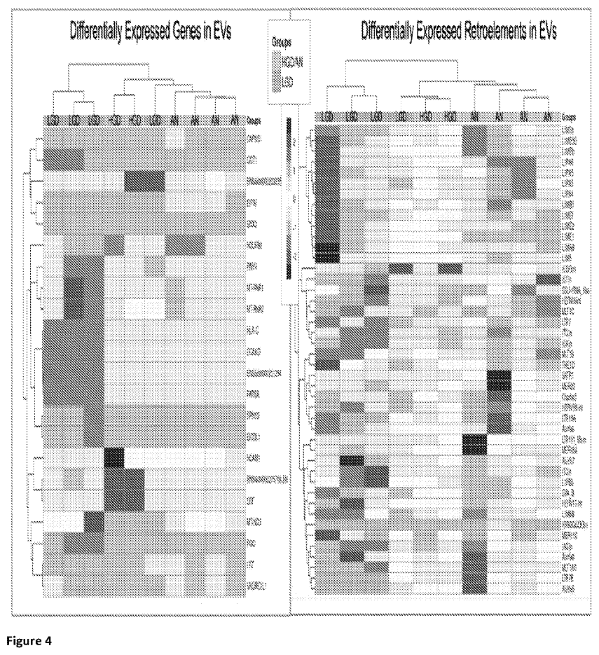

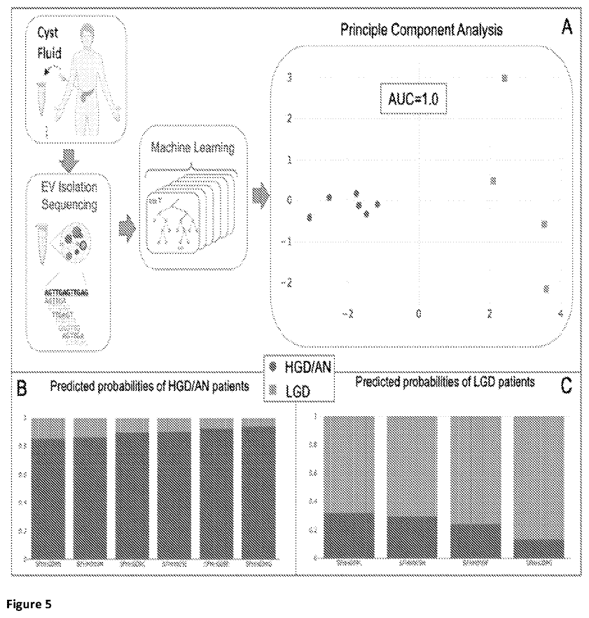

Results A striking pattern of differential expression in both gene transcripts and retroelements packaged inside of EVs (FIG. 4) was shown. A variety of RNA gene transcripts inside of EVs that are differentially expressed between LGD and HGD/AN were discovered. As illustrated in FIG. 4 we observed a clear shift in expression of RNA fragments mapping to specific genes that group to low-grade, high-grade and AN.

Retroelements were discovered packaged inside of EVs that are associated with PCL grade, specific LINE-1 elements are significantly upregulated in HGD/AN patients, and specific SVA, Alu and HERVs are down regulated (FIG. 5). This observation in PCL mirrors the findings from the serum of glioma patients (e.g., Example 1) where retroelements were observed packaged inside of EV's that predict glioma subtype (See, FIG. 2, 3). A k-mer and random forest based machine learning (ML) was applied to create a predictive model for classifying LGD vs. HGD/AN. Due to the small sample HGD and AN were grouped as this is the most clinically relevant classifier. It was shown that using just 10 subjects perfect predictive power (AUC=1.0) was achieved to delineate LGD vs HGD/AN. This model is robust to outliers based on results from leave one out 10-fold cross validation. A ML analyses with k-mer lengths from 11-31 bp were run and all showed the same perfect prediction accuracy. The number of differentially expressed mers are striking, for example with k set to 31 bp 9133 mers were observed that provide information to the ML model. However, it is important to note that ˜60 mers have a feature importance above 15 that contribute substantially to the predictive model. The top performing mers identified by ML map to LINE-1 elements.

### REFERENCES

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art. In case of a conflict between the specification and any of the incorporated references, the specification (including any amendments thereof, which may be based on an incorporated reference), shall control. Standard art-accepted meanings of terms are used herein unless indicated otherwise. Standard abbreviations for various terms are used herein.

Although the foregoing subject matter has been described in some detail by way of illustration and example for purposes of clarity of understanding, it will be understood by those skilled in the art that certain changes and modifications can be practiced within the scope of the appended claims.
1 Ostrom Q T, Cote D J, Ascha M, Kruchko C, Barnholtz-Sloan J S. Adult Glioma Incidence and Survival by Race or Ethnicity in the United States From 2000 to 2014. JAMA Oncol. 2018; 4 (9): 1254-62. Epub 2018 Jun. 23. doi: 10.1001/jamaoncol.2018.1789. PubMed PMID: 29931168; PMCID: PMC6143018.
2 Eckel-Passow J E, Lachance D H, Molinaro A M, Walsh K M, Decker P A, Sicotte H, Pekmezci M, Rice T, Kosel M L, Smirnov I V, Sarkar G, Caron A A, Kollmeyer T M, Praska C E, Chada A R, Halder C, Hansen H M, McCoy L S, Bracci P M, Marshall R, Zheng S, Reis G F, Pico A R, O'Neill B P, Buckner J C, Giannini C, Huse J T, Perry A, Tihan T, Berger M S, Chang S M, Prados M D, Wiemels J, Wiencke J K, Wrensch M R, Jenkins R B. Glioma Groups Based on 1p/19q, IDH, and TERT Promoter Mutations in Tumors. N Engl J Med. 2015;372 (26): 2499-508. Epub 2015 Jun. 11. doi: 10.1056/NEJMoa1407279. PubMed PMID: 26061753; PMCID: PMC4489704.
3 Eckel-Passow J E, Drucker K L, Kollmeyer T M, Kosel M L, Decker P A, Molinaro A M, Rice T, Praska C E, Clark L, Caron A, Abyzov A, Batzler A, Song J S, Pekmezci M, Hansen H M, McCoy L S, Bracci P M, Wiemels J, Wiencke J K, Francis S, Burns T C, Giannini C, Lachance D H, Wrensch M, Jenkins R B. Adult diffuse glioma GWAS by molecular subtype identifies variants in D2HGDH and FAM20C. Neuro Oncol. 2020; 22 (11): 1602-13. Epub 2020 May 10. doi: 10.1093/neuonc/noaa117. PubMed PMID: 32386320; PMCID: PMC7690366.
4 Molinaro A M, Taylor J W, Wiencke J K, Wrensch M R. Genetic and molecular epidemiology of adult diffuse glioma. Nat Rev Neurol. 2019; 15 (7): 405-17. Epub 2019 Jun. 23. doi: 10.1038/s41582-019-0220-2. PubMed PMID: 31227792; PMCID: PMC7286557.
5 Parvez K, Parvez A, Zadeh G. The diagnosis and treatment of pseudoprogression, radiation necrosis and brain tumor recurrence. Int J Mol Sci. 2014; 15 (7): 11832-46. Epub 2014 Jul. 6. doi: 10.3390/ijms150711832. PubMed PMID: 24995696; PMCID: PMC4139817.
6 Wen P Y, Macdonald D R, Reardon D A, Cloughesy T F, Sorensen A G, Galanis E, Degroot J, Wick W, Gilbert M R, Lassman A B, Tsien C, Mikkelsen T, Wong E T, Chamberlain M C, Stupp R, Lamborn K R, Vogelbaum M A, van den Bent M J, Chang S M. Updated response assessment criteria for high-grade gliomas: response assessment in neuro-oncology working group. J Clin Oncol. 2010; 28 (11): 1963-72. Epub 2010 Mar. 17. doi: 10.1200/JCO.2009.26.3541. PubMed PMID: 20231676.
7 Thust S C, van den Bent M J, Smits M. Pseudoprogression of brain tumors. J Magn Reson Imaging. 2018. Epub 2018 May 8. doi: 10.1002/jmri.26171. PubMed PMID: 29734497; PMCID: PMC6175399.
8 Elshafeey N, Kotrotsou A, Hassan A, Elshafei N, Hassan I, Ahmed S, Abrol S, Agarwal A, El Salek K, Bergamaschi S, Acharya J, Moron F E, Law M, Fuller G N, Huse J T, Zinn P O, Colen R R. Multicenter study demonstrates radiomic features derived from magnetic resonance perfusion images identify pseudoprogression in glioblastoma. Nat Commun. 2019; 10 (1): 3170. Epub 2019 Jul. 20. doi: 10.1038/s41467-019-11007-0. PubMed PMID: 31320621; PMCID: PMC6639324.
9 Tom M C, Park D Y J, Yang K, Leyrer C M, Wei W, Jia X, Varra V, Yu J S, Chao S T, Balagamwala E H, Suh J H, Vogelbaum M A, Barnett G H, Prayson R A, Stevens G H J, Peereboom D M, Ahluwalia M S, Murphy E S. Malignant Transformation of Molecularly Classified Adult Low-Grade Glioma. Int J Radiat Oncol Biol Phys. 2019; 105 (5): 1106-12. Epub 2019 Aug. 29. doi: 10.1016/j.ijrobp.2019.08.025. PubMed PMID: 31461674.
10 Oberheim Bush N A, Chang S. Treatment Strategies for Low-Grade Glioma in Adults. 10 J Oncol Pract. 2016; 12 (12): 1235-41. Epub 2016 Dec. 13. doi: 10.1200/JOP.2016.018622. PubMed PMID: 27943684.
11 Buckner J C, Shaw E G, Pugh S L, Chakravarti A, Gilbert M R, Barger G R, Coons S, Ricci P, Bullard D, Brown P D, Stelzer K, Brachman D, Suh J H, Schultz C J, Bahary J P, Fisher B J, Kim H, Murtha A D, Bell E H, Won M, Mehta M P, Curran W J, Jr. Radiation plus Procarbazine, CCNU, and Vincristine in Low-Grade Glioma. N Engl J Med. 2016; 374 (14): 1344-55. Epub 2016 Apr. 7. doi: 10.1056/NEJMoa1500925. PubMed PMID: 27050206; PMCID: PMC5170873.
12 Hunter C, Smith R, Cahill D P, Stephens P, Stevens C, Teague J, Greenman C, Edkins S, Bignell G, Davies H, O'Meara S, Parker A, Avis T, Barthorpe S, Brackenbury L, Buck G, Butler A, Clements J, Cole J, Dicks E, Forbes S, Gorton M, Gray K, Halliday K, Harrison R, Hills K, Hinton J, Jenkinson A, Jones D, Kosmidou V, Laman R, Lugg R, Menzies A, Perry J, Petty R, Raine K, Richardson D, Shepherd R, Small A, Solomon H, Tofts C, Varian J, West S, Widaa S, Yates A, Easton D F, Riggins G, Roy J E, Levine K K, Mueller W, Batchelor T T, Louis D N, Stratton M R, Futreal P A, Wooster R. A hypermutation phenotype and somatic MSH6 mutations in recurrent human malignant gliomas after alkylator chemotherapy. Cancer Res. 2006;66 (8): 3987-91. Epub 2006 Apr. 19. doi: 10.1158/0008-5472.CAN-06-0127. PubMed PMID: 16618716; PMCID: PMC7212022.
13 Margison G P, Santibanez Koref M F, Povey A C. Mechanisms of carcinogenicity/chemotherapy by 06-methylguanine. Mutagenesis. 2002;17 (6): 483-7. Epub 2002 Nov. 19. doi: 10.1093/mutage/17.6.483. PubMed PMID: 12435845.
14 Bodell W J, Gaikwad N W, Miller D, Berger M S. Formation of DNA adducts and induction of lacI mutations in Big Blue Rat-2 cells treated with temozolomide: implications for the treatment of low-grade adult and pediatric brain tumors. Cancer Epidemiol Biomarkers Prev. 2003; 12 (6): 545-51. Epub 2003 Jun. 20. PubMed PMID: 12815001.
15 Choi S, Yu Y, Grimmer M R, Wahl M, Chang S M, Costello J F. Temozolomide-associated hypermutation in gliomas. Neuro Oncol. 2018; 20 (10): 1300-9. Epub 2018 Feb. 17. doi: 10.1093/neuonc/noy016. PubMed PMID: 29452419; PMCID: PMC6120358.
16 Yip S, Miao J, Cahill D P, Iafrate A J, Aldape K, Nutt C L, Louis D N. MSH6 mutations arise in glioblastomas during temozolomide therapy and mediate temozolomide resistance. Clin Cancer Res. 2009; 15 (14): 4622-9. Epub 2009 Jul. 9. doi: 10.1158/1078-0432.CCR-08-3012. PubMed PMID: 19584161; PMCID: PMC2737355.
17 Cahill D P, Codd P J, Batchelor T T, Curry W T, Louis D N. MSH6 inactivation and emergent temozolomide resistance in human glioblastomas. Clin Neurosurg. 2008; 55:165-71. Epub 2008 Jan. 1. PubMed PMID: 19248684.
18 Touat M, Li Y Y, Boynton A N, Spurr L F, Iorgulescu J B, Bohrson C L, Cortes-Ciriano I, Birzu C, Geduldig J E, Pelton K, Lim-Fat M J, Pal S, Ferrer-Luna R, Ramkissoon S H, Dubois F, Bellamy C, Currimjee N, Bonardi J, Qian K, Ho P, Malinowski S, Taquet L, Jones R E, Shetty A, Chow K H, Sharaf R, Pavlick D, Albacker L A, Younan N, Baldini C, Verreault M, Giry M, Guillerm E, Ammari S, Beuvon F, Mokhtari K, Alentorn A, Dehais C, Houillier C, Laigle-Donadey F, Psimaras D, Lee E Q, Nayak L, McFaline-Figueroa J R, Carpentier A, Cornu P, Capelle L, Mathon B, Barnholtz-Sloan J S, Chakravarti A, Bi W L, Chiocca E A, Fehnel K P, Alexandrescu S, Chi S N, Haas-Kogan D, Batchelor T T, Frampton G M, Alexander B M, Huang R Y, Ligon A H, Coulet F, Delattre J Y, Hoang-Xuan K, Meredith D M, Santagata S, Duval A, Sanson M, Cherniack A D, Wen P Y, Reardon D A, Marabelle A, Park P J, Idbaih A, Beroukhim R, Bandopadhayay P, Bielle F, Ligon K L. Mechanisms and therapeutic implications of hypermutation in gliomas. Nature. 2020; 580 (7804): 517-23. Epub 2020 Apr. 24. doi: 10.1038/s41586-020-2209-9. PubMed PMID: 32322066.
19 Del Bene M, Osti D, Faletti S, Beznousenko G V, DiMeco F, Pelicci G. Extracellular vesicles: the key for precision medicine in glioblastoma. Neuro Oncol. 2021. Epub 2021 Sep. 29. doi: 10.1093/neuonc/noab229. PubMed PMID: 34581817.
20 Dard-Dascot C, Naquin D, d'Aubenton-Carafa Y, Alix K, Thermes C, van Dijk E. Systematic comparison of small RNA library preparation protocols for next-generation sequencing. BMC Genomics. 2018; 19 (1): 118. Epub 2018 Feb. 7. doi: 10.1186/s12864-018-4491-6. PubMed PMID: 29402217; PMCID: PMC5799908.
21 Yang J K, Yang J P, Tong J, Jing S Y, Fan B, Wang F, Sun G Z, Jiao B H. Exosomal miR-221 targets DNM3 to induce tumor progression and temozolomide resistance in glioma. J Neurooncol. 2017;131 (2): 255-65. Epub 2016 Nov. 12. doi: 10.1007/s11060-016-2308-5. PubMed PMID: 27837435.
22 Wang K, Wang X, Zou J, Zhang A, Wan Y, Pu P, Song Z, Qian C, Chen Y, Yang S, Wang Y. miR-92b controls glioma proliferation and invasion through regulating Wnt/beta-catenin signaling via Nemo-like kinase. Neuro Oncol. 2013; 15 (5): 578-88. Epub 2013 Feb. 19. doi: 10.1093/neuonc/not004. PubMed PMID: 23416699; PMCID: PMC3635522.
23 Ebrahimkhani S, Vafaee F, Hallal S, Wei H, Lee M Y T, Young P E, Satgunaseelan L, Beadnall H, Barnett M H, Shivalingam B, Suter C M, Buckland M E, Kaufman K L. Deep sequencing of circulating exosomal microRNA allows non-invasive glioblastoma diagnosis. NPJ Precis Oncol. 2018; 2:28. Epub 2018 Dec. 20. doi: 10.1038/s41698-018-0071-0. PubMed PMID: 30564636; PMCID: PMC6290767.
24 Song X, Zhang N, Han P, Moon B S, Lai R K, Wang K, Lu W. Circular RNA profile in gliomas revealed by identification tool UROBORUS. Nucleic Acids Res. 2016; 44 (9): e87. Epub 2016 Feb. 14. doi: 10.1093/nar/gkw075. PubMed PMID: 26873924; PMCID: PMC4872085.
25 Kong Y, Rose C M, Cass A A, Williams A G, Darwish M, Lianoglou S, Haverty P M, Tong A J, Blanchette C, Albert M L, Mellman I, Bourgon R, Greally J, Jhunjhunwala S, Chen-Harris H. Transposable element expression in tumors is associated with immune infiltration and increased antigenicity. Nat Commun. 2019; 10 (1): 5228. Epub 2019 Nov. 21. doi: 10.1038/s41467-019-13035-2. PubMed PMID: 31745090; PMCID: PMC6864081.
26 Ishak C A, Carvalho D D D. Reactivation of Endogenous Retroelements in Cancer Development and Therapy. Annual Review of Cancer Biology. 2020; 4 (1): 159-76. doi: 10.1146/annurev-cancerbio-030419-033525.
27 Lorenzi C, Barriere S, Villemin J P, Dejardin Bretones L, Mancheron A, Ritchie W. iMOKA: k-mer based software to analyze large collections of sequencing data. Genome Biol. 2020; 21 (1): 261. Epub 2020 Oct. 15. doi: 10.1186/s13059-020-02165-2. PubMed PMID: 33050927; PMCID: PMC7552494.
28 Balaj L, Lessard R, Dai L, Cho Y J, Pomeroy S L, Breakefield X O, Skog J. Tumour microvesicles contain retrotransposon elements and amplified oncogene sequences. Nat Commun. 2011; 2:180. Epub 2011 Feb. 3. doi: 10.1038/ncomms1180. PubMed PMID: 21285958; PMCID: PMC3040683.
29 Turchinovich A, Surowy H, Serva A, Zapatka M, Lichter P, Burwinkel B. Capture and Amplification by Tailing and Switching (CATS). An ultrasensitive ligation-independent method for generation of DNA libraries for deep sequencing from picogram amounts of DNA and RNA. RNA Biol. 2014; 11 (7): 817-28. Epub 2014 Jun. 13. doi: 10.4161/rna.29304. PubMed PMID: 24922482; PMCID: PMC4179956.
30 Langdon W B. Performance of genetic programming optimised Bowtie2 on genome comparison and analytic testing (GCAT) benchmarks. BioData Min. 2015;8 (1): 1. Epub 2015 Jan. 27. doi: 10.1186/s13040-014-0034-0. PubMed PMID: 25621011; PMCID: PMC4304608.
31 Dobin A, Davis C A, Schlesinger F, Drenkow J, Zaleski C, Jha S, Batut P, Chaisson M, Gingeras T R. STAR: ultrafast st universal RNA-seq aligner. Bioinformatics. 2013; 29 (1): 15-21. Epub 2012 Oct. 30. doi: 10.1093/bioinformatics/bts635. PubMed PMID: 23104886; PMCID: PMC3530905.
32 Du Y, Huang Q, Arisdakessian C, Garmire L X. Evaluation of STAR and Kallisto on Single Cell RNA-Seq Data Alignment. G3 (Bethesda). 2020; 10 (5): 1775-83. Epub 2020 Mar. 30. doi: 10.1534/g3.120.401160. PubMed PMID: 32220951; PMCID: PMC7202009.
33 Buchfink B, Xie C, Huson D H. Fast and sensitive protein alignment using DIAMOND. Nat Methods. 2015;12 (1): 59-60. Epub 2014 Nov. 18. doi: 10.1038/nmeth.3176. PubMed PMID: 25402007.
34 Love M I, Huber W, Anders S. Moderated estimation of fold change and dispersion for RNA-seq data with DESeq2. Genome Biol. 2014; 15 (12): 550. Epub 2014 Dec. 18. doi: 10.1186/s13059-014-0550-8. PubMed PMID: 25516281; PMCID: PMC4302049.
35 Zhang X O, Dong R, Zhang Y, Zhang J L, Luo Z, Zhang J, Chen L L, Yang L. Diverse alternative back-splicing and alternative splicing landscape of circular RNAs. Genome Res. 2016; 26 (9): 1277-87. Epub 2016 Jul. 2. doi: 10.1101/gr.202895.115. PubMed PMID: 27365365; PMCID: PMC5052039

1. A method for (i) detecting or determining the presence, type, classification, or grade of a tumor, cyst, lesion, mass, cancer, or any combination thereof, or (ii) classifying or subtyping a tumor, cyst, lesion, mass, cancer, or any combination thereof in a sample obtained from a subject, the method comprising:
a. generating a RNA sequence library from the subject using capture and amplification by tailing and switching (CATS) from RNA isolated from extracellular vesicles obtained from a serum sample of a subject suspected of having or having a tumor, cyst, lesion, mass, cancer, or any combination thereof, wherein the RNA sequence library comprises at one retroelement, transposable element, full or partial RNA transcript, non-coding RNA, or any combination thereof, from the RNA isolated from the extracellular vesicles;
b. providing a processing system comprising a computer processor and a non-transitory computer memory comprising a database and at least one k-mers based machine learning algorithm, wherein the k-mers based machine learning algorithm is configured to:
i. apply the machine learning algorithm to the RNA sequence library generated in step a) to generate k-mers results for the subject; and
ii. use the k-mers results from the subject and a reference k-mers profile obtained from a control group to generate a set of probabilities to indicate whether the k-mers results from the subject are statistically similar to an outcome of interest, wherein the outcome of interest is to (i) identify the presence of a tumor, cyst, lesion, mass, cancer, or any combination thereof in the subject; (ii) determine the type or grade of tumor, cyst, lesion, mass, or cancer in the subject; (iii) classify the tumor cyst, lesion, mass, cancer, or any combination thereof; (iv) determine the subtype of tumor, cyst, lesion, mass, cancer, or any combination thereof in the subject; or (v) any combination of (i)-(iv); and
c. detecting or determining the presence, type, or grade of tumor, cyst, lesion, mass, and/or cancer, or classification or subtype of tumor, cyst, lesion, mass and/or cancer in the subject based on the probabilities generated in step b).

2. The method of claim 1, wherein the tumor is a brain tumor.

3. The method of claim 2, wherein the brain tumor is a glioma.

4. The method of claim 3, wherein the glioma is an astrocytoma, glioblastoma, or oligodendroglioma.

5. The method of claim 1, wherein the method further comprises obtaining a serum sample from the subject and isolating the extracellular vesicles in the serum sample, plasma sample, or cyst fluid sample obtained from the subject.

6. The method of claim 1, wherein the retroelements or transposable elements are long interspersed nuclear elements (LINE), short interspersed nuclear elements (SINE), SINE-VNTR-Alu (SVA), long terminal repeat (LTR) retroelements, non-LTR elements, Tyrosine recombinase (YR) retroelements, Penelope like elements (PLEs), pericentromeric satellites, alpha satellites, or any combination thereof.

7. The method of claim 1, wherein the CATS library preparation is modified by utilizing polyethylene glycol molecular crowding to increase the efficiency of RNA sequencing.

8. The method of claim 7, wherein the modified CATS library preparation utilizes unique molecular identifiers (UMI), where random base pairs are synthesized on sequence adapters to aid in direct quantification of the RNA template.

9. The method of claim 1, wherein the cyst is a pancreatic cyst.

10. A method for monitoring progression or recurrence of a tumor, cyst, lesion, mass, cancer, or any combination in a subject, the method comprising:
a. generating a RNA sequence library using capture and amplification by tailing and switching (CATS) from RNA isolated from extracellular vesicles obtained from a serum sample of a subject having a tumor, cyst, lesion, mass, cancer, or any combination thereof; wherein the RNA sequence library comprises at least one retroelement, transposable element, full or partial RNA transcript, non-coding RNA, or any combination thereof, from the RNA isolated from the extracellular vesicles;
b. providing a processing system comprising a computer processor and a non-transitory computer memory comprising a database and at least one k-mers based machine learning algorithm, wherein the k-mers based machine learning algorithm is configured to:
i. apply the machine learning algorithm to the RNA sequence library generated in step a) to generate k-mers results for the subject; and
ii. use the k-mers results from the subject and a reference k-mers profile obtained from a control group to generate a set of probabilities to indicate whether the k-mers results from the subject are statistically similar to an outcome of interest, wherein the outcome of interest is to identify (i) whether the tumor, cyst, lesion, mass, cancer, or any combination thereof in the subject has increased in size; or (ii) has recurred or re-appeared in the subject; and
c. determining whether (i) the tumor, cyst, lesion, mass, cancer, or any combination thereof in the subject has increased in size and progressed; or (ii) the tumor, cyst, lesion, mass, cancer, or any combination thereof has reoccurred or re-appeared in the subject based on the probabilities generated in step b).

11. The method of claim 10, wherein the method further comprises predicting the survival of the subject based on the determination of whether the tumor, cyst, lesion, mass, cancer, or any combination thereof has or has not progressed in the subject.

12. The method of claim 1, wherein the tumor is a brain tumor.

13. The method of claim 12, wherein the brain tumor is a glioma.

14. The method of claim 13, wherein the glioma is an astrocytoma, glioblastoma, or oligodendroglioma.

15. The method of claim 10, wherein the method further comprises obtaining a serum sample from the subject and isolating the extracellular vesicles in the serum sample, plasma sample, or cyst fluid sample obtained from the subject.

16. The method of claim 10, wherein the retroelements or transposable elements are long interspersed nuclear elements (LINE), short interspersed nuclear elements (SINE), SINE-VNTR-Alu (SVA), long terminal repeat (LTR) retroelements, non-LTR elements, Tyrosine recombinase (YR) retroelements, Penelope like elements (PLEs), pericentromeric satellites, alpha satellites, or any combination thereof.

17. The method of claim 10, wherein the CATS library preparation is modified by utilizing polyethylene glycol molecular crowding to increase the efficiency of RNA sequencing.

18. The method of claim 17, wherein the modified CATS utilizes unique molecular identifiers (UMI), where random base pairs are synthesized on sequence adapters to aid in direct quantification of the RNA template.

19. The method of claim 10, wherein the cyst is a pancreatic cyst.

20. The method of claim 10, wherein the subject is being administered at least one therapeutic agent to treat the tumor, cyst, lesion, mass, cancer, or any combination thereof.

21. A method for diagnosing a glioma in a subject, the method comprising:
a. generating ‘a RNA sequence library using capture and amplification by tailing and switching (CATS) from RNA isolated from extracellular vesicles obtained from a serum sample of a subject; wherein the RNA sequence library comprises at least one retroelement, transposable element, full or partial RNA transcript, non-coding RNA, or any combination thereof, from the RNA isolated from the extracellular vesicles;
b. providing a processing system comprising a computer processor and a non-transitory computer memory comprising a database and at least one k-mers based machine learning algorithm, wherein the k-mers based machine learning algorithm is configured to:
i. apply the machine learning algorithm to the RNA sequence library generated in step a) to generate k-mers results for the subject; and
ii. use the k-mers results from the subject and a reference k-mers profile obtained from a control group to generate a set of probabilities to indicate whether the k-mers results from the subject are statistically similar to an outcome of interest, wherein the outcome of interest is to identify the presence or absence of a glioma; and
c. determining whether or not the subject has a glioma based on the probabilities generated in step b).

22. The method of claim 21, wherein the glioma is an astrocytoma, glioblastoma, or oligodendroglioma.

23. The method of claim 21, wherein the method further comprises obtaining a serum sample from the subject and isolating the extracellular vesicles in the serum sample obtained from the subject.

24. The method of claim 21, wherein the retroelements or transposable elements are long interspersed nuclear elements (LINE), short interspersed nuclear elements (SINE), SINE-VNTR-Alu (SVA), long terminal repeat (LTR) retroelements, non-LTR elements, Tyrosine recombinase (YR) retroelements, Penelope like elements (PLEs), pericentromeric satellites, alpha satellites, or any combination thereof.

25. The method of claim 21, wherein the CATS library preparation is modified by utilizing polyethylene glycol molecular crowding to increase the efficiency of RNA sequencing.

26. The method of claim 25, wherein the modified CATS utilizes unique molecular identifiers (UMI), where random base pairs are synthesized on sequence adapters to aid in direct quantification of the RNA template.

27. The method of claim 21, wherein the subject is suspected of having a tumor, cyst, lesion, mass, cancer, or any combination thereof.

28. A system for (i) detecting or determining the presence, type, or grade of a tumor, cyst, lesion, mass, and/or cancer; or (ii) classifying or subtyping a tumor, cyst, lesion, mass, and/or cancer, the system comprising:
a. a RNA sequence library using capture and amplification by tailing and switching (CATS) from RNA isolated from extracellular vesicles obtained from a serum sample, plasma sample, or cyst fluid sample of a subject of interest having a tumor, cyst, lesion, mass, cancer, or any combination thereof, wherein the RNA sequence library comprises at least one or more retroelements, transposable elements, full or partial RNA transcripts, non-coding RNAs, or any combination thereof, from the RNA isolated from the extracellular vesicles;
b. a k-mers based machine learning algorithm for analyzing the one or more retroelements, transposable elements, or combination thereof, from the RNA sequence library; and
c. a reference database generated from a control group for detecting or determining the presence, type, or grade of the tumor, cyst, lesion, mass, and/or cancer, or classifying or subtyping the tumor, cyst, lesion, mass, and/or cancer in the sample based on the analysis in step b).

29. The system of claim 28, wherein the tumor is a brain tumor.

30. The system of claim 29, wherein the brain tumor is a glioma.

31. The system of claim 30, wherein the glioma is an astrocytoma, glioblastoma or oligodendroglioma.

32. The system of claim 28, wherein the retroelements or transposable elements are long interspersed nuclear elements (LINE), short interspersed nuclear elements (SINE), SINE-VNTR-Alu (SVA), long terminal repeat (LTR) retroelements, non-LTR elements, Tyrosine recombinase (YR) retroelements, Penelope like elements (PLEs), pericentromeric satellites, alpha satellites, or any combination thereof.

33. The system of claim 28, wherein the CATS library preparation is modified by utilizing polyethylene glycol molecular crowding to increase the efficiency of RNA sequencing.

34. The system of claim 33, wherein the modified CATS library preparation utilizes unique molecular identifiers (UMI), where random base pairs are synthesized on sequence adapters to aid in direct quantification of the RNA template.

35. The system of claim 28, wherein the cyst is a pancreatic cyst.

36. A method of improving the accuracy of determining whether a subject is at risk of developing a glioma or a recurrence of a glioma, the method comprising the steps:
a. generating a sequence library from RNA isolated from extracellular vesicles obtained from a serum sample of a subject, wherein the sequence library comprises RNA of one or more retroelements, transposable elements, full or partial RNA transcripts, non-coding RNAs, or any combination thereof, obtained from the extracellular vesicles using capture and amplification by tailing and switching (CATS) and one or more unique molecular identifiers;
b. aligning the sequences of the sequence library generated in step a) with a reference genome sequence;
c. providing a processing system comprising a computer processor and a non-transitory computer memory comprising a database and at least one k-mers based machine learning algorithm, wherein the k-mers based machine learning algorithm is configured to:
i. apply the machine learning algorithm to the sequences aligned in step b) to generate k-mers results for the subject; and
ii. use the k-mers results from the subject and a reference k-mers profile obtained from a control group to generate a set of probabilities to indicate whether the k-mers results from the subject are statistically similar to an outcome of interest, wherein the outcome of interest is to identify (i) whether the subject is at risk of developing a glioma; or (ii) reoccurrence or re-appearance of a glioma; and
d. determining whether (i) the subject is at risk of a glioma; or (ii) whether or not a glioma has reoccurred or re-appeared in the subject based on the probabilities generated in step c.

37. The method of claim 36, wherein the reference genome sequence is hg38 or hg19.

38. The method of claim 36, wherein the glioma is an astrocytoma, glioblastoma, or oligodendroglioma.

39. The method of claim 36, wherein the retroelements or transposable elements are long interspersed nuclear elements (LINE), short interspersed nuclear elements (SINE), SINE-VNTR-Alu (SVA), long terminal repeat (LTR) retroelements, non-LTR elements, Tyrosine recombinase (YR) retroelements, Penelope like elements (PLEs), pericentromeric satellites, alpha satellites, or any combination thereof.

40. The method of claim 36, wherein the CATS library preparation is modified by utilizing polyethylene glycol molecular crowding to increase the efficiency of RNA sequencing.

41. The method of claim 40, wherein the modified CATS library preparation utilizes unique molecular identifiers (UMI), where random base pairs are synthesized on sequence adapters to aid in direct quantification of the RNA template.
